(19) United States
(12) Patent Application Publication
(10) Pub. No.: US 2025/0204866 A1
(43) Pub. Date: Jun. 26, 2025

(54) APPARATUS AND A METHOD FOR A PLURALITY OF TIME SERIES DATA

(71) Applicant: Anumana, Inc., Cambridge, MA (US)

(72) Inventors: Anand Ramani, Fresno, CA (US); Rohit Jain, Danville, CA (US); Leon Ptaszek, Milton, MA (US); Kappagantula Gopalakrishna Murty, Nanaimo (CA); Mukesh Kamath Bola, Bengaluru (IN); Yogisha Heggadahalli Jayendra, Bengaluru (IN); Sanjeev Shrinivas Nadapurohit, Bengaluru (IN); Karthik K. Bharadwaj, Bengaluru (IN); Animesh Agarwal, San Mateo, CA (US)

(73) Assignee: Anumana, Inc., Cambridge, MA (US)

(21) Appl. No.: 18/786,066

(22) Filed: Jul. 26, 2024

Related U.S. Application Data
(60) Provisional application No. 63/614,864, filed on Dec. 26, 2023.

(51) Int. Cl. A61B 5/00; A61B 5/283; A61B 5/349

(52) U.S. Cl. CPC A61B 5/7264; A61B 5/283; A61B 5/349

(57) ABSTRACT

An apparatus for labeling a plurality of time series data is disclosed. The apparatus includes at least processor and a memory communicatively connected to the processor. The memory instructs the processor to receive a plurality of time series data. The memory instructs the processor to generate a plurality of time series segments for each time series represented within the plurality of time series data. The memory instructs the processor to identify one or more segment attributes for each time series segment. The memory instructs the processor to classify each time series segment of the plurality of time series segments to at least one time series label as a function of the one or more segment attributes. The memory instructs the processor to generate at least one labeled time series segment for each time series segment of the plurality of time series segments as a function of the classification.

### CROSS-REFERENCE TO RELATED APPLICATIONS

This application claims the benefit of priority of U.S. Provisional Patent Application Ser. No. 63/614,864, filed on Dec. 26, 2023, and titled “METHOD AND SYSTEM FOR LABELING OF INTRACARDIAC ELECTROGRAMS,” which is incorporated by reference herein in its entirety.

### FIELD OF THE INVENTION

The present invention generally relates to the field of medical technology. In particular, the present invention is directed to an apparatus and a method for labeling a plurality of time series data.

### BACKGROUND

Monitoring time series data is crucial to patient care. Intracardiac electrograms and Electrocardiograms provide critical information associated with an electrical activity of the heart of a patient, aiding in the diagnosis of cardiac abnormalities, for example, arrhythmias, ischemia, and myocardial infraction. However, to interpret the critical information from the intracardiac electrograms and Electrocardiograms, an identification of different segments of the intracardiac electrogram is required.

### SUMMARY OF THE DISCLOSURE

In an aspect, an apparatus for the labeling a plurality of time series data is disclosed. The memory instructs the processor to receive a plurality of time series data. The memory instructs the processor to generate a plurality of time series segments for each time series represented within the plurality of time series data. The memory instructs the processor to identify one or more segment attributes for each time series segment of the plurality of time series segments. The memory instructs the processor to classify each time series segment of the plurality of time series segments to at least one time series label as a function of the one or more segment attributes. The classification includes training a time series classifier using time series training data, wherein time series training data comprises examples of segment attributes correlated to examples of time series labels. The classification also includes classifying each time series segment of the plurality of time series segments to at least one time series label using the trained time series classifier. The memory instructs the processor to generate at least one labeled time series segment for each time series segment of the plurality of time series segments as a function of the classification.

In another aspect, a method for the labeling a plurality of time series data is disclosed. The method includes receiving, using at least a processor, a plurality of time series data. The method includes generating, using the at least a processor, a plurality of time series segments for each time series represented within the plurality of time series data. The method includes identifying, using the at least a processor, one or more segment attributes for each time series segment of the plurality of time series segments. The method includes classifying, using the at least a processor each time series segment of the plurality of time series segments to at least one time series label as a function of the one or more segment attributes. The classification includes training a time series classifier using time series training data, wherein time series training data comprises examples of segment attributes correlated to examples of time series labels. The classification also includes classifying each time series segment of the plurality of time series segments to at least one time series label using the trained time series classifier. The method include generating, using the at least a processor, at least one labeled time series segment for each time series segment of the plurality of time series segments as a function of the classification

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for labeling a plurality of time series data;

FIG. 2 is a flow diagram of an exemplary implementation of a machine learning pipeline for generating a label for labelling an intracardiac electrogram according to one embodiment;

FIGS. 3A-F are exemplary embodiments of labeling strategies for generating labels corresponding to a plurality of intracardiac electrogram;

FIG. 4 is a block diagram of an exemplary embodiment of training a machine learning model used for labeling of the time-series data;

FIG. 5 is a block diagram of an exemplary machine-learning process;

FIG. 6 is a block diagram of an exemplary embodiment of a time series database;

FIG. 7 is a diagram of an exemplary embodiment of a neural network;

FIG. 8 is a diagram of an exemplary embodiment of a node of a neural network;

FIG. 9 is an illustration of an exemplary embodiment of fuzzy set comparison;

FIG. 10 is an illustration of an exemplary labeled time series segment;

FIG. 11 is a flow diagram of an exemplary method for labeling a plurality of time series data; and

FIG. 12 is a block diagram of a computing system that can be used to implement any one or more of the methodologies disclosed herein and any one or more portions thereof.

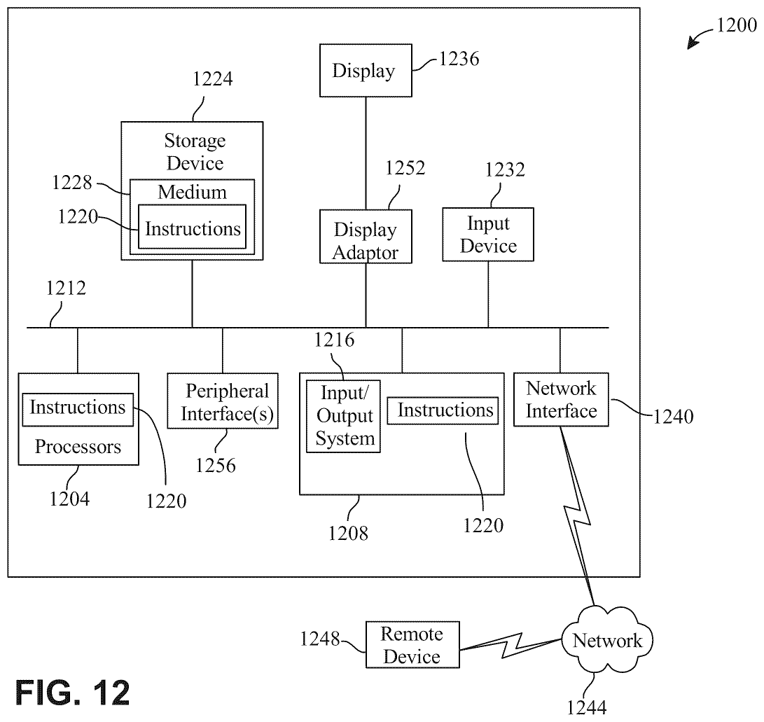

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus and a method for labeling a plurality of time series data. The memory instructs the processor to receive a plurality of time series data. The memory instructs the processor to generate a plurality of time series segments for each time series represented within the plurality of time series data. The memory instructs the processor to identify one or more segment attributes for each time series segment of the plurality of time series segments. The memory instructs the processor to classify each time series segment of the plurality of time series segments to at least one time series label as a function of the one or more segment attributes. The classification includes training a time series classifier using time series training data, wherein time series training data comprises examples of segment attributes correlated to examples of time series labels. The classification also includes classifying each time series segment of the plurality of time series segments to at least one time series label using the trained time series classifier. The memory instructs the processor to generate at least one labeled time series segment for each time series segment of the plurality of time series segments as a function of the classification. Exemplary embodiments illustrating aspects of the present disclosure are described below in the context of several specific examples.

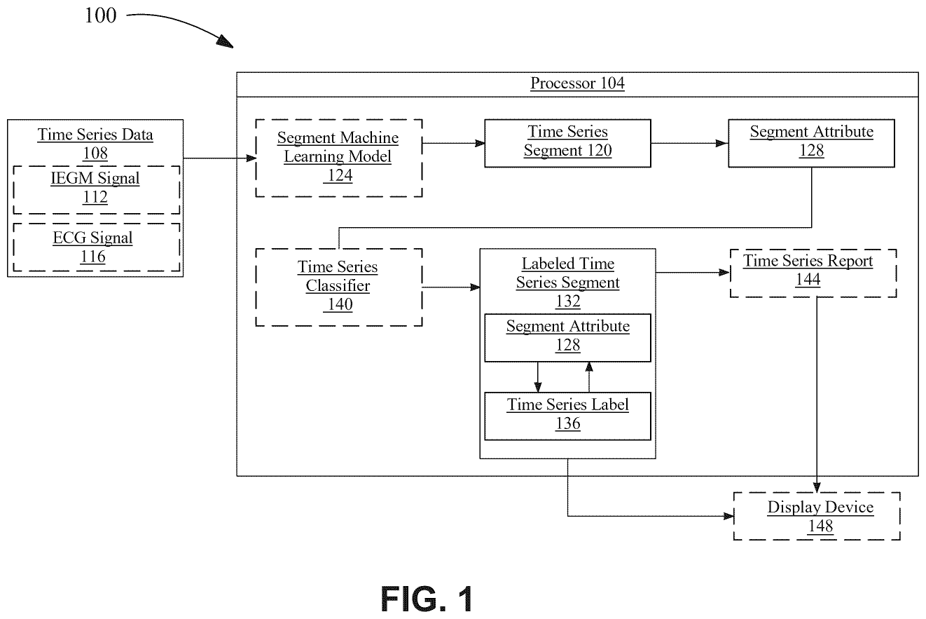

Referring now to FIG. 1, an exemplary embodiment of an apparatus 100 for labeling a plurality of time series data is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 includes a memory. Memory is communicatively connected to processor 104. Memory may contain instructions configuring processor 104 to perform tasks disclosed in this disclosure. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, apparatus, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example, and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, without limitation, via a bus or other facility for intercommunication between elements of a computing device. Communicative connecting may also include indirect connections via, for example, and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, processor 104 is configured to receive a plurality of time series data 108. As used in the current disclosure, “time series data” refers to any data that is indexed in time order. Time series data 108 may be a continuous sequence of data points collected or recorded at successive points in time, typically at uniform intervals. The time series data 108 may include a plurality of intracardiac electrogram (IEGM) data 112 from a user. As used in the current disclosure, “intracardiac electrogram (IEGM) data” is a signal representative of the electrical activity recorded within the heart. IEGM data 112 may include detailed information about the local electrical events in specific heart chambers or regions. IEGM data 112 may consist of several distinct waves and segments, each representing different phases of the cardiac cycle and localized electrical activities. These segments may include atrial electrograms, ventricular electrograms, and specific potentials such as Pulmonary Vein Potentials (PVPs). Atrial electrograms may represent the electrical activity associated with atrial depolarization, while ventricular electrograms may correspond to ventricular depolarization and repolarization. PVPs are critical in the context of atrial fibrillation, indicating electrical activity in the pulmonary veins. The attributes of these segments, such as amplitude, duration, and morphology, provide vital information for diagnosing and treating various cardiac conditions, including arrhythmias and conduction abnormalities. In an embodiment, each catheter or sensor placed within the heart may generate individual IEGM data 112, which can be processed and analyzed to support clinical decision-making during electrophysiological studies and ablation procedures.

With continued reference to FIG. 1, processor 104 may be configured to receive time series data from a variety of sources, including de-identified case data. This de-identified case data may encompass time series data 108 that has been removed of patient identifiers. This may be done to ensure patient privacy while enabling comprehensive analysis. The de-identified nature of the case data ensures that personal identifiers are removed, allowing the data to be used for research and analysis without compromising patient confidentiality. By utilizing de-identified case data, the system can process and analyze information from numerous patients, enhancing the robustness and reliability of the clinical decision-making process during electrophysiological studies and ablation procedures.

With continued reference to FIG. 1, time series data 108 may include a plurality of electrocardiogram (ECG) data 116 from a user. As used in the current disclosure, a “electrocardiogram data” is a signal representative of the electrical activity of the heart. The ECG data 116 may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves provide information about the duration and regularity of various phases of the cardiac cycle. The ECG data 116 can be used to help diagnose various heart conditions, such as arrhythmias, myocardial infarction (heart attack), conduction abnormalities, electrolyte imbalances, coronary heart disease, and the like. In an embodiment, each sensor may generate an individual ECG data 116.

With continued reference to FIG. 1, time series data 108 may include a plurality of time series data related to cardiac care. This may include electrocardiogramaignals, surface cardiac signals, catheter signals, cardiac computed tomography (CT) signals, cardiac magnetic resonance imaging (MRI) signals, echocardiography signals, and various ablation delivery signals. Catheter signals, such as those measuring positional stability, temperature, and contact force, offer detailed information about the conditions within the heart during invasive procedures. Cardiac CT and MRI signals provide high-resolution images of the heart's structure and function, helping to identify anatomical abnormalities. Echocardiography signals use ultrasound waves to create real-time images of the heart, allowing for the assessment of cardiac function and blood flow. Ablation delivery signals, including those from radiofrequency (RF), cryoablation (Cryo), and pulsed-field ablation (PFA) procedures, are critical for monitoring and guiding therapeutic interventions to treat arrhythmias.

With continued reference to FIG. 1, processor 104 may be configured to receive time series data 108 using an application programming interface (API). As used herein, an “application programming interface” is a set of functions that allow applications to access data and interact with external software components, operating systems, or microdevices, such as another web application or computing device. An API may define the methods and data formats that applications can use to request and exchange information. APIs enable seamless integration and functionality between different systems, applications, or platforms. An API may deliver time series data 108 to apparatus 100 from a system/application that is associated with a user, medical provider, medical facility, or other third-party custodian of user information. In an embodiment, an API may be configured to query for web applications or other websites to retrieve time series data 108 to. An API may be further configured to filter through web applications according to a filter criterion. In this disclosure, “filter criteria” are conditions the web applications must fulfill in order to qualify for API. Web applications may be filtered based off these filter criteria. Filter criterion may include, without limitation, web application dates, web application traffic, web application types, web applications addresses, and the like. Once an API filters through web applications according to a filter criterion, it may select a web application. Processor 104 may transmit, through the API, time series data 108 to apparatus 100. API may further automatically fill out user entry fields of the web application with the user credentials in order to gain access to the time series data 108 to. Web applications may include, without limitation, a medical database, hospital website, file scanning, email programs, third party websites, governmental websites, or the like.

With continued reference to FIG. 1, time series data 108 to may be generated from one or more medical records. As used in the current disclosure, a “medical record” is a document that contains information regarding the user's medical history. Medical records may include data about any medical procedures, medical tests, medical images, observations of a medical professional, prescription history, diagnostic history, government records (i.e., birth certificates, social security cards, and the like), and the like of the user. Medical records may be identified using a web crawler. Medical records may include a variety of types of “notes” entered over time by a medical professional. Medical records may be converted into machine-encoded text using an optical character reader (OCR).

Still referring to FIG. 1, in some embodiments, optical character recognition or optical character reader (OCR) includes automatic conversion of images of written (e.g., typed, handwritten, or printed text) into machine-encoded text. In some cases, recognition of at least a keyword from an image component may include one or more processes, including without limitation optical character recognition (OCR), optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine learning processes. In some cases, intelligent word recognition (IWR) may recognize written text, one word at a time, for instance by employing machine learning processes.

Still referring to FIG. 1, in some cases, OCR may be an “offline” process, which analyses a static document or image frame. In some cases, handwriting movement analysis can be used as input for handwriting recognition. For example, instead of merely using shapes of glyphs and words, this technique may capture motions, such as the order in which segments are drawn, the direction, and the pattern of putting the pen down and lifting it. This additional information can make handwriting recognition more accurate. In some cases, this technology may be referred to as “online” character recognition, dynamic character recognition, real-time character recognition, and intelligent character recognition.

Still referring to FIG. 1, in some cases, OCR processes may employ pre-processing of image components. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to the image component to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from the background of the image component. In some cases, binarization may be required for example if an employed OCR algorithm only works on binary images. In some cases, a line removal process may include the removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify a script allowing an appropriate OCR algorithm to be selected. In some cases, a character isolation or “segmentation” process may separate signal characters, for example, character-based OCR algorithms. In some cases, a normalization process may normalize the aspect ratio and/or scale of the image component.

Still referring to FIG. 1, in some embodiments, an OCR process will include an OCR algorithm. Exemplary OCR algorithms include matrix-matching process and/or feature extraction processes. Matrix matching may involve comparing an image to a stored glyph on a pixel-by-pixel basis. In some cases, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at the same scale as input glyph. Matrix matching may work best with typewritten text.

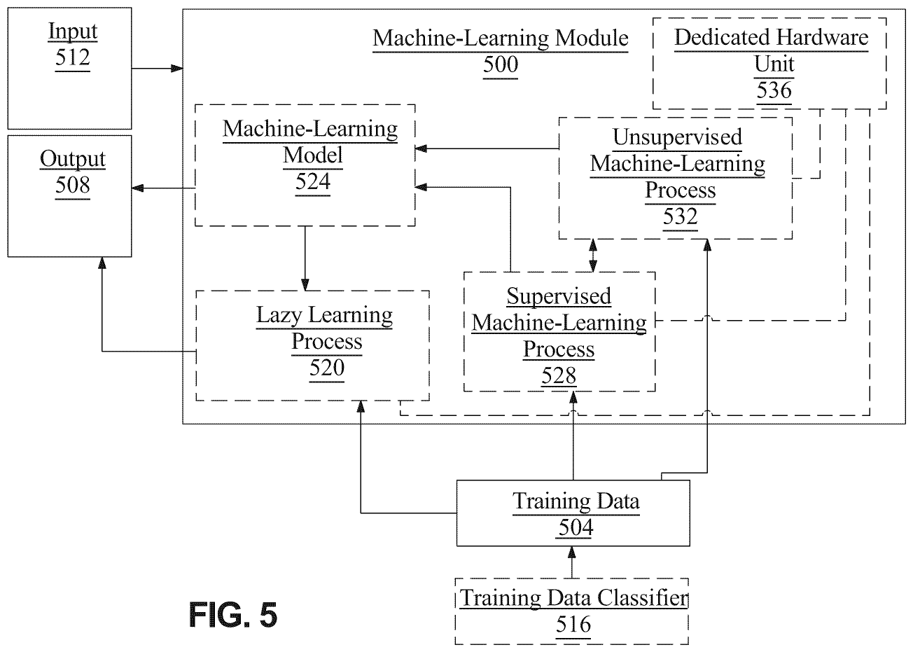

Still referring to FIG. 1, in some embodiments, an OCR process may include a feature extraction process. In some cases, feature extraction may decompose a glyph into features. Exemplary non-limiting features may include corners, edges, lines, closed loops, line direction, line intersections, and the like. In some cases, feature extraction may reduce dimensionality of representation and may make the recognition process computationally more efficient. In some cases, extracted features can be compared with an abstract vector-like representation of a character, which might reduce to one or more glyph prototypes. General techniques of feature detection in computer vision are applicable to this type of OCR. In some embodiments, machine-learning processes like nearest neighbor classifiers (e.g., k-nearest neighbors algorithm) can be used to compare image features with stored glyph features and choose a nearest match. OCR may employ any machine-learning process described in this disclosure, for example machine-learning processes described with reference to FIGS. 5-7. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

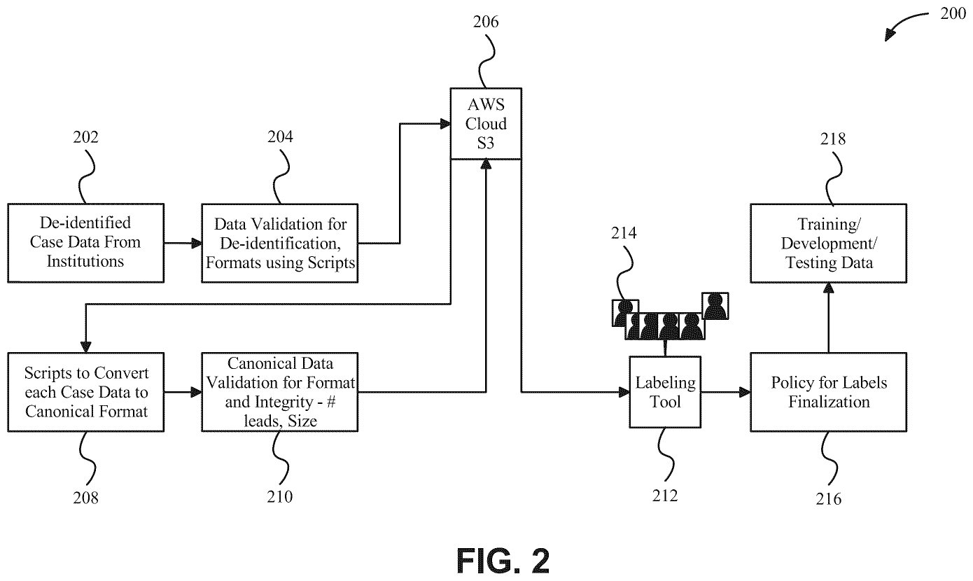

Still referring to FIG. 1, in some cases, OCR may employ a two-pass approach to character recognition. The second pass may include adaptive recognition and use letter shapes recognized with high confidence on a first pass to recognize better remaining letters on the second pass. In some cases, two-pass approach may be advantageous for unusual fonts or low-quality image components where visual verbal content may be distorted. Another exemplary OCR software tool include OCRopus. OCRopus development is led by German Research Centre for Artificial Intelligence in Kaiserslautern, Germany. In some cases, OCR software may employ neural networks, for example neural networks as taught in reference to FIGS. 2, 4, and 5.

Still referring to FIG. 1, in some cases, OCR may include post-processing. For example, OCR accuracy can be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make use of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

With continued reference to FIG. 1, the time series data 108 may be generated using at least a sensor. As used in this disclosure, a “sensor” is a device that is configured to detect an input and/or a phenomenon and transmit information related to the detection. Sensor may detect a plurality of data. A plurality of data detected by sensor may include, but is not limited to, electrocardiogram data 112, time series data 108, IEGM data 112, electrical signals related to the heart, and the like. In one or more embodiments, and without limitation, sensor may include a plurality of sensors.

With continued reference to FIG. 1, at least a sensor may include an ECG machine. In one or more embodiments, and without limitation, sensor may include one or more electrodes, and the like. Electrodes used for an electrocardiogram (ECG) are small sensors or conductive patches that are placed on specific locations on the body to detect and record the electrical signals generated by the heart. Senor serves as the interface between the body and the ECG machine, allowing for the measurement and recording of the heart's electrical activity. A plurality of sensors may include 10 electrodes used for a standard 12-lead ECG, placed in specific positions on the chest and limbs of the patient. Sensors may also include various lead systems including, 1-lead, 2-lead, 6-leads, 12, leads, standard limb leads, augmented limb leads, pectoral leads, and the like. These electrodes are typically made of a conductive material, such as metal or carbon, and are connected to lead wires that transmit the electrical signals to the ECG machine for recording. Proper electrode placement may be crucial to ensure accurate signal detection and recording. A number of electrodes used by an ECG machine may depend on a particular machine in use and may vary from a single electrode on a wearable device to twelve or more electrodes, or any number in between.

With continued reference to FIG. 1, the plurality of sensors may be placed on each limb, wherein there may be at least one sensor on each arm and/or leg of the user. These sensors may be labeled I, II, III, V1, V2, V3, V4, V5, V6, and the like. For example, Sensor I may be placed on the left arm, Sensor II may be placed on the right arm, and Sensor III may be placed on the left leg. Additionally, a plurality of sensors may be placed on various portions of the patient's torso and chest. For example, a sensor V1 may be placed in the fourth intercostal space at both the right sternal borders and sensor V2 may be fourth intercostal space at both the left sternal borders. A sensor V3 may also be placed between sensors V2 and V4, halfway between their positions. Sensor V4 may be placed in the fifth intercostal space at the midclavicular line. Sensor V5 may be placed horizontally at the same level as sensor V4 but in the anterior axillary line. Sensor V6 may be placed horizontally at the same level as V4 and V5 but in the midaxillary line.

With continued reference to FIG. 1, the plurality of sensors may include augmented unipolar sensors. These sensors may be labeled as aVR, aVL, and aVF. These sensor may be derived from the limb sensors and provide additional information about the heart's electrical activity. These leads are calculated using specific combinations of the limb leads and help assess the electrical vectors in different orientations. For example, aVR may be derived from Sensor II and Sensor III. In another example, aVL may be derived from sensor I and Sensor III. Additionally, aVF may be derived from Lead I and Lead II. The combination of limb sensors, precordial sensors, and augmented unipolar sensors allows for a comprehensive assessment of the heart's electrical activity in three dimensions.

With continued reference to FIG. 1, the intracardiac electrogram (IEGM) data 112 may be generated using at least a catheter. As used in this disclosure, a “catheter” is a device that is configured to detect electrical activity within the heart and transmit information related to this activity. The catheter may detect a plurality of data. A plurality of data detected by the catheter may include, but is not limited to, IEGM data 112, electrical signals related to specific regions of the heart, and the like. In one or more embodiments, and without limitation, a catheter may include a plurality of electrodes positioned along its length.

With continued reference to FIG. 1, at least a catheter may include an electrophysiology (EP) catheter. In one or more embodiments, and without limitation, the catheter may include one or more electrodes, mapping electrodes, and the like. Electrodes used for intracardiac recordings are small sensors embedded in the catheter that detect and record the electrical signals generated by the heart from within its chambers. The catheter may serve as the interface between the heart's internal environment and the recording system, allowing for the measurement and recording of localized electrical activity. A plurality of electrodes may be arranged along the catheter to capture detailed and specific intracardiac signals. These electrodes are typically made of a conductive material, such as platinum or iridium, and are connected to lead wires that transmit the electrical signals to the recording system for processing. Proper catheter placement is crucial to ensure accurate signal detection and recording. The number of electrodes on a catheter may vary depending on the particular catheter in use and may range from a few to several dozen, depending on the mapping and ablation needs.

With continued reference to FIG. 1, the plurality of electrodes on the catheter may be positioned at various strategic locations within the heart. For example, electrodes may be placed in the right atrium, right ventricle, left atrium, left ventricle, and pulmonary veins. These electrodes are labeled based on their position and function, such as His-bundle electrodes for detecting signals from the His bundle, or coronary sinus electrodes for signals from the coronary sinus. Additionally, the electrodes may be placed at various positions to create a three-dimensional map of the heart's electrical activity during procedures like catheter ablation. For example, electrodes may be positioned in the right atrial appendage, the left atrial appendage, and around the pulmonary vein ostia to capture detailed electrical activity from these regions.

With continued reference to FIG. 1, the plurality of electrodes may also include specialized electrodes for different types of mapping and ablation procedures. These may include circular mapping catheters for creating comprehensive electrical maps of the atria, or contact force-sensing catheters that provide feedback on the pressure applied to the heart tissue during ablation. These catheters are connected to advanced electrophysiology recording systems that amplify, filter, and process the signals for real-time visualization and analysis. The combination of various catheter types and electrode configurations allows for a detailed and comprehensive assessment of the heart's electrical activity, aiding in the diagnosis and treatment of complex arrhythmias.

With continued reference to FIG. 1, preprocessing the plurality of time series data 108 may be done using one or more digital filtering techniques to enhance the quality of the recorded signals. As used in the current disclosure, “digital filtering techniques” are methods used to manipulate and refine digital signals to remove unwanted components. This may include removing noise or artifacts, while preserving the essential features of the signal. These techniques may ensure that the time series data 108 is accurate and reliable for subsequent analysis and segmentation. A digital filtering technique may include the application of mathematical algorithms to the raw data to isolate and remove specific frequency components. For example, low-pass filters may allow signals with frequencies below a certain threshold to pass through while attenuating higher frequency noise. Conversely, high-pass filters permit high-frequency signals to pass while reducing the impact of lower frequency interference. Band-pass filters combine these principles to isolate a specific range of frequencies, which is particularly useful for focusing on the relevant portions of the cardiac signal. Examples of digital filtering techniques may include the use of Finite Impulse Response (FIR) filters, which apply a finite sequence of weights to the signal. FIR filters are known for their stability and linear phase response, making them ideal for applications requiring precise timing, such as ECG and IEGM data analysis. Another technique is Infinite Impulse Response (IIR) filters, which use feedback to create a response that can theoretically last indefinitely. IIR filters are efficient in terms of computational resources and are widely used for real-time signal processing. Additionally, adaptive filtering techniques, such as the least mean squares (LMS) algorithm, can dynamically adjust their parameters in response to changes in the signal characteristics. This adaptability makes them particularly effective in environments with varying noise levels. Wavelet transform is another advanced filtering technique that decomposes the signal into components at different scales, allowing for the selective filtering of noise while retaining important signal features. By applying these digital filtering techniques, processor 104 can effectively preprocess the plurality of time series data 108, removing noise and artifacts that could otherwise obscure the critical information within the cardiac signals. This preprocessing step ensures that the subsequent analysis, segmentation, and interpretation of the data are based on high-quality, accurate signals, ultimately improving the reliability and effectiveness of cardiac diagnostics and interventions.

With continued reference to FIG. 1, the system may be configured to generate a plurality of time series segments 120 for each time series represented within the plurality of time series data. As used in this disclosure, “time series segments” refer to discrete portions of the overall time series data that are analyzed individually. Each segment may correspond to a specific interval of time and captures a particular aspect of the data within that interval. The generation of these time series segments involves dividing the continuous time series data into smaller, manageable parts. For instance, the intracardiac electrogram (IEGM) data 112, these segments may represent various phases of the cardiac cycle, such as atrial depolarization, ventricular depolarization, and ventricular repolarization. Each segment can be defined by its start and end points, duration, and the specific attributes it contains, such as amplitude, frequency, and morphology. The process of generating these segments may involve the use of algorithms to identify and extract meaningful segments from the continuous data stream. This can be based on predefined criteria, such as specific signal characteristics or timing intervals. In an embodiment, processor 104 may annotate each time series segment 120 as a function of the portion of the time series that is represented. each segment based on its characteristics and relevance to the overall analysis. For example, labeling segments as P-wave, QRS complex, or T-wave in ECG data.

With continued reference to FIG. 1, processor 104 may apply feature extraction algorithms to identify key attributes of the signal, such as peaks, troughs, and intervals that are indicative of specific phases of the cardiac cycle. The feature extraction process may include identifying he highest points (peaks) and the lowest points (troughs) in the time series data. Peaks in the IEGM data 112 might represent critical points such as the R-wave in the QRS complex, which signifies ventricular depolarization. Similarly, troughs can correspond to points like the Q-wave and S-wave, which are crucial for accurate characterization of the QRS complex. Processor 104 may measure intervals between these key points to further delineate phases of the cardiac cycle. For example, the PR interval (from the start of the P-wave to the start of the QRS complex) reflects the time taken for the electrical impulse to travel from the atria to the ventricles. The QT interval (from the start of the QRS complex to the end of the T-wave) provides information about the total time for ventricular depolarization and repolarization. Accurately measuring these intervals is essential for diagnosing various cardiac conditions, such as prolonged QT syndrome.

With continued reference to FIG. 1, processor 104 may employ pattern recognition models. These models may be trained on vast datasets of annotated cardiac signals to recognize specific patterns associated with different cardiac events. For instance, the pattern recognition model may differentiate between normal sinus rhythms and abnormal rhythms like atrial fibrillation by analyzing the consistency and morphology of the P-wave and R-R intervals. The supervised pattern recognition models may use labeled data to improve the classification accuracy of each detected segment, ensuring that the identified segments conform to predefined medical criteria. The pattern recognition model may identify time series segments 120 by first preprocessing the continuous ECG or IEGM data to enhance signal quality. This preprocessing may include filtering out noise and artifacts that could interfere with accurate pattern recognition. The model may employ feature extraction techniques to isolate key attributes of the signal, such as peak amplitudes, waveform shapes, and inter-peak intervals. For example, in identifying the QRS complex, the model looks for the rapid succession of the Q wave, R wave, and S wave, characterized by their distinct high amplitude and short duration. Once these features are extracted, the pattern recognition model may compare them against the known patterns in its training dataset. This may be done using machine learning algorithms such as convolutional neural networks (CNNs) or recurrent neural networks (RNNs). The model may process the features in context, recognizing the sequential nature of the cardiac cycle. For example, the model can identify a P-wave by its lower amplitude and rounded shape preceding a QRS complex, and a T-wave by its broader, rounded shape following the QRS complex. In an embodiment, the model's ability to recognize patterns extends to complex arrhythmias as well. For instance, in detecting atrial fibrillation, the model may analyze the variability and irregularity in the R-R intervals, coupled with the absence of distinct P-waves. The model's trained understanding of these patterns allows it to segment the time series data accurately, even in the presence of noise or atypical signal presentations. In an embodiment, the pattern recognition model may continuously refine its segmentation accuracy through supervised learning. By using labeled training data, where each segment of the time series is annotated with its correct classification, the model adjusts its internal parameters to minimize classification errors. This iterative process involves comparing its predictions against the labeled data, identifying discrepancies, and updating its pattern recognition criteria to better align with the predefined medical standards.

With continued reference to FIG. 1, processor 104 may employ temporal alignment methods to synchronize the segments across multiple channels of data, such as recordings from different electrodes within the heart. This synchronization may be useful for constructing a comprehensive view of the cardiac activity, as it ensures that the timing and relationship between signals from different parts of the heart are accurately represented. Temporal alignment methods can account for slight variations in signal timing due to differences in electrode placement or signal propagation delays, thus providing a coherent multi-channel dataset for analysis. The temporal alignment process may include the identification of common reference points across all channels. These reference points could be distinct features in the cardiac cycle, such as the peak of the R-wave in the QRS complex, which is a prominent and easily identifiable marker. By aligning these reference points across multiple recordings, the processor can ensure that all channels are temporally synchronized to a common timeline. Processor 104 may employ cross-correlation techniques to measure the time lag between signals from different electrodes. By calculating the cross-correlation function, the processor can determine the degree of similarity between signals at various time shifts, identifying the optimal alignment that maximizes the correlation. This method helps in adjusting for delays caused by the physical distance between electrodes and the time it takes for electrical impulses to propagate through the heart tissue. In an embodiment, dynamic time warping (DTW) may be employed to handle non-linear variations in signal timing. DTW is particularly useful when the timing differences are not uniform across the entire recording. It allows the processor to stretch or compress segments of the time series to achieve the best possible alignment. This technique ensures that even in the presence of complex timing discrepancies, the signals can be accurately synchronized.

With continued reference to FIG. 1, once the signals are aligned, the processor can segment the synchronized data into consistent and meaningful time series segments 120. This alignment may enable a more accurate and holistic analysis of the heart's electrical activity, as it allows the simultaneous examination of multi-channel data. For example, synchronized segments from different electrodes can reveal the propagation of electrical impulses through the heart, aiding in the identification of conduction pathways and regions of interest for ablation. Temporal alignment also facilitates the comparison of recordings from different sessions or patients. By standardizing the timing across datasets, the processor can aggregate and analyze data on a larger scale, identifying patterns and trends that might not be apparent in isolated recordings. This capability is essential for longitudinal studies and comparative analysis, enhancing the understanding of cardiac behavior over time and across diverse patient populations.

With continued reference to FIG. 1, once the segments are accurately identified and classified, the segmented data may be annotated with metadata, including time stamps, segment labels, and relevant physiological metrics. These annotations may provide a structured framework for further clinical analysis and decision-making. Time stamps indicate the precise moments when each cardiac event occurs, segment labels categorize the types of cardiac events (e.g., P-wave, QRS complex, T-wave), and physiological metrics (such as amplitude and duration) offer quantitative measures of each segment's characteristics. This detailed annotation process is essential for diagnosing complex cardiac conditions and planning appropriate therapeutic interventions.

With continued reference to FIG. 1, processor 104 may identify time series segments 120 using a segment machine-learning model 124. As used in the current disclosure, a “segment machine-learning model” is a machine-learning model that is configured to generate time series segments 120. Segment machine-learning model 124 may be consistent with the machine-learning model described below in FIG. 2. Inputs to the segment machine-learning model 124 may include time series data 108, IEGM data 112, ECG data 116, examples of time series segments 120, and the like. Outputs to the segment machine-learning model 124 may include time series segments 120 tailored to the time series data 108. Segment training data may include a plurality of data entries containing a plurality of inputs that are correlated to a plurality of outputs for training a processor by a machine-learning process. In an embodiment, segment training data may include a plurality of time series data 108 correlated to examples of time series segments 120. Segment training data may be received from database 600. Segment training data may contain information about time series data 108, IEGM data 112, ECG data 116, examples of time series segments 120, and the like. In an embodiment, segment training data may be iteratively updated as a function of the input and output results of past segment machine-learning model 124 or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, the training data for a segment machine-learning model 124 may consist of a comprehensive and meticulously curated dataset of annotated time series signals. This dataset may include various recordings of electrocardiograms (ECGs) and intracardiac electrograms (IEGMs) from a diverse population of patients, encompassing a wide range of cardiac conditions and normal heart function. Each recording in the training data may be labeled with precise annotations identifying key segments of the cardiac cycle, such as the P-wave, QRS complex, T-wave, and any relevant pathological features like arrhythmias or conduction blocks. The annotations may be performed by expert cardiologists who ensure the accuracy and reliability of the labels. Alternatively, the annotations may be examples of past labeled time series data that has been output from apparatus 100. The dataset may be enriched with metadata, including patient demographics, clinical history, and any relevant procedural details, providing a rich context for the ML model to learn from. The variability in the training data, including different heart rates, signal noise levels, and anatomical variations, helps the model generalize well across different scenarios. This robust and diverse training dataset enables the ML model to accurately identify and segment time series data, ensuring its effectiveness in real-world clinical applications.

With continued reference to FIG. 1, processor 104 may identify one or more segment attributes 128 for each time series segment 120 of the plurality of time series segments. As used in the current disclosure, a “segment attribute” refers to specific characteristics or features of the time series data that define and distinguish each segment. These attributes are crucial for accurately identifying and classifying different phases of the cardiac cycle and other significant events in the time series data. Processor 104 may apply signal processing techniques to the time series segments 120 to extract fundamental segment attributes 128 such as amplitude, duration, frequency, and morphology. Amplitude may measure the height of the signal peaks and troughs, indicating the strength of the electrical activity. Duration may refer to the length of time each segment occupies, providing information on the timing of different cardiac events. Frequency analysis, often performed using techniques like Fast Fourier Transform (FFT), may reveal the dominant frequencies within a time series segments 120, which can help identify specific types of arrhythmias or other abnormalities. In some cases, processor 104 employ a morphology analysis. This may involve examining the shape and structure of the waveform within each segment. For example, processor 104 may analyze the characteristic shapes of the P-wave, QRS complex, and T-wave in ECG data to distinguish between them. This may include identifying the presence of sharp peaks, broad waves, or notches that signify different electrical activities in the heart. The processor may use advanced pattern recognition algorithms and machine learning models trained on annotated datasets to accurately classify these morphological features. Additionally, processor 104 may compute more complex attributes such as the slope of the signal (rate of change of amplitude), the area under the curve (integral of the signal over time), and the presence of specific patterns like the PR interval, QT interval, and R-R interval. These attributes provide deeper insights into the temporal dynamics and the physiological implications of the cardiac cycle. By leveraging these extracted attributes, processor 104 can accurately identify and annotate each time series segment 120, ensuring precise segmentation and classification. This detailed attribute identification process is essential for diagnosing cardiac conditions, guiding treatment decisions, and conducting further clinical analysis.

With continued reference to FIG. 1, processor 104 may generate an attribute score as a function of each segment attribute. As used in the current disclosure, an “attribute score” refers to a quantification of the significance or quality of a specific segment attribute. These scores may be used to evaluate and compare the characteristics of different time series segments, enabling more accurate identification and classification of cardiac events. Processor 104 may generate an attribute score by first extracting the fundamental segment attributes such as amplitude, duration, frequency, and morphology from each time series segment 120. For each attribute, the processor may apply one or more signal processing techniques to calculate precise numerical values. For instance, the amplitude score may be derived from the peak-to-peak measurement of the signal, indicating the strength of the electrical activity. The duration score is calculated based on the length of time each segment occupies, reflecting the timing of cardiac events. Frequency analysis, often utilizing Fast Fourier Transform (FFT), produces a frequency score that highlights the dominant frequencies within the segment, which can be crucial for identifying arrhythmias. Morphology analysis may include examining the shape and structure of the waveform within each segment. The morphology score may consider factors such as the sharpness of peaks, the breadth of waves, and the presence of notches. Advanced machine learning models, trained on annotated datasets, enhance the accuracy of this analysis, ensuring that each morphological feature is correctly identified and scored. Additionally, processor 104 may compute more complex attributes such as the slope of the signal, the area under the curve, and the presence of specific patterns like the PR interval, QT interval, and R-R interval. Each of these attributes may be quantified with a corresponding score. The slope score may reflect the rate of change of amplitude, providing insights into the rapidity of electrical conduction. The area under the curve score measures the total electrical activity over time, and pattern scores for intervals provide information on the temporal dynamics of the cardiac cycle. Generating these attribute scores may be done using a combination of statistical analysis, machine learning algorithms, and heuristic rules. For example, a machine learning model might be trained to assign scores based on labeled training data, where each segment attribute's importance and quality have been pre-determined by expert clinicians. The model can learn to weigh different attributes according to their relevance for specific cardiac conditions, allowing for a nuanced and accurate scoring system. By leveraging these attribute scores, processor 104 can effectively rank and prioritize different time series segments based on their clinical significance. This scoring system aids in the precise segmentation and classification of the data, facilitating accurate diagnosis, guiding treatment decisions, and supporting further clinical research.

With continued reference to FIG. 1, an attribute score may be normalized to bring all attributes, such as time series data 108, health conditions, genomic traits, and the like, onto a comparable scale. This step is crucial to eliminate any bias introduced by different units or measurement scales. Normalization techniques can include min-max scaling, z-score normalization, or logarithmic transformation. In an embodiment, an attribute score may be expressed as a numerical score, a linguistic value, or an alphabetical score. A non-limiting example of a numerical score may include a scale from 0-1, 1-10, 1-100, 1-1000, and the like, where a rating of 1 may represent minimal impact on the user's heart health, while a rating of 10 may represent a symptom or condition with a significant impact on the user's heart health. In another non-limiting example, linguistic values may include descriptors such as “No Impact,” “Moderate Impact,” “Significant Impact,” and the like. An attribute score may also indicate whether the impact on the user's heart health is positive or negative, which can be represented using a combination of negative and positive values. In some embodiments, linguistic values may correspond to a specific score range. For example, a condition or symptom that receives a score between 40-60 on a scale from 1-100 may be classified as having a “Moderate Impact.” This approach ensures that all attribute scores are comparable, providing a standardized method for assessing the impact of various factors on heart health.

With continued reference to FIG. 1, processor 104 may utilize a combination of statistical analysis, machine learning algorithms, and heuristic rules. For example, a machine learning model might be trained to assign scores based on labeled training data, where each segment attribute's importance and quality have been pre-determined by expert clinicians. The model can learn to weigh different attributes according to their relevance for specific cardiac conditions, allowing for a nuanced and accurate scoring system. The annotated training dataset may include each time series segment labeled with its corresponding cardiac event and scored for its attributes by medical professionals or a previous iteration of apparatus 100. This dataset serves as the foundation for training the machine learning model. During training, the model learns to recognize patterns and relationships between raw time series data and their corresponding attribute scores. Techniques such as supervised learning are employed, where the model is iteratively refined by minimizing the error between its predicted scores and the actual scores provided in the training data. Furthermore, statistical analysis plays a crucial role in refining the attribute scores. Descriptive statistics such as mean, median, standard deviation, and variance are calculated to understand the distribution of each attribute within the dataset. These statistics may be used to normalize the scores and ensure that they are comparable across different segments and patients. For instance, if the amplitude of the QRS complex varies significantly across patients, normalization ensures that the amplitude score remains consistent and meaningful. In an embodiment, heuristic rules, derived from clinical knowledge and experience, may also be incorporated into the scoring process. These rules may provide a framework for interpreting the attribute scores within the context of known physiological and pathological conditions. For example, a heuristic rule might state that a PR interval longer than 200 milliseconds typically indicates first-degree atrioventricular block. Such rules help in setting thresholds and reference values that guide the assignment of attribute scores.

With continued reference to FIG. 1, segment attributes 128 encompass various characteristics of each time series segment 120, providing critical insights into the cardiac cycle. One primary attribute is the amplitude, which indicates the strength of the electrical signal, such as the height of the R-wave in the QRS complex. Another important attribute is the duration, measuring the time span of each segment, such as the length of the PR interval, which reflects the time taken for the electrical impulse to travel from the atria to the ventricles. Frequency attributes involve analyzing the dominant frequencies within a segment, helping to identify specific arrhythmias by revealing patterns in the heart's electrical activity. Morphology may be another segment attribute 128, detailing the shape and structure of the waveform. For instance, the rounded shape of the P-wave signifies atrial depolarization, while the sharp, rapid succession of the QRS complex indicates ventricular depolarization. The slope, or the rate of change of the signal's amplitude, provides information about the speed of electrical conduction within the heart. The area under the curve, which is the integral of the signal over time, offers insights into the overall energy of the electrical activity within that segment. Furthermore, temporal attributes such as the PR interval, QT interval, and R-R interval may be useful for understanding the timing relationships within the cardiac cycle. The PR interval measures the time from the start of the P-wave to the start of the QRS complex, indicating atrioventricular conduction time. The QT interval spans from the beginning of the QRS complex to the end of the T-wave, representing the total time for ventricular depolarization and repolarization. The R-R interval, the time between successive R-waves, is a measure of the heart rate and its variability.

With continued reference to FIG. 1, processor 104 may generate segment attributes 128 using an attribute machine-learning model. As used in the current disclosure, a “attribute machine-learning model” is a machine-learning model that is configured to generate segment attributes 128. Attribute machine-learning model may be consistent with the machine-learning model described below in FIG. 2. Inputs to the attribute machine-learning model may include time series data 108, IEGM data 112, ECG data 116, time series segment 120, examples of segment attributes 128, and the like. Outputs to the attribute machine-learning model may include segment attributes 128 tailored to the time series segments 120. Attribute training data may include a plurality of data entries containing a plurality of inputs that are correlated to a plurality of outputs for training a processor by a machine-learning process. In an embodiment, attribute training data may include a plurality of time series segments 120 correlated to examples of segment attributes 128. Attribute training data may be received from database 600. Attribute training data may contain information about time series data 108, IEGM data 112, ECG data 116, time series segment 120, examples of segment attributes 128, and the like. In an embodiment, attribute training data may be iteratively updated as a function of the input and output results of past attribute machine-learning model or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, processor 104 may be configured to generate at least one labeled time series segment 132 for each time series segment 120 of the plurality of time series segments. As used in the current disclosure, a “labeled time series segment” refers to a discrete portion of the time series data that has been identified, classified, and annotated with specific labels based on its characteristics. This process involves analyzing the segment to determine its significance within the context of the cardiac cycle or other physiological events and assigning a label that accurately describes this significance. A labeled time series segment 132. A labeled time series segment 132 may include labeled data points within the segment, representing a specific interval of time in the continuous time series data. For instance, in the context of ECG or IEGM data, the segment data might capture the electrical activity of the heart during a single cardiac event, such as a P-wave, QRS complex, or T-wave. Generating at least one labeled time series segment 132 may include employing one or more pattern recognition models trained on vast datasets of annotated cardiac signals. These models may be capable of recognizing specific patterns associated with different phases of the cardiac cycle and various cardiac events. For example, the models can differentiate between normal sinus rhythms and abnormal rhythms such as atrial fibrillation by analyzing the consistency and morphology of the P-wave, QRS complex, and T-wave, as well as the R-R intervals. The pattern recognition models may be used to each labeled time series segment 132 based on these learned patterns, assigning appropriate labels that reflect the identified cardiac events. The classification step is discussed in greater detail herein below. After the classification step, processor 104 may perform temporal alignment to ensure synchronization across multiple channels of data. This involves aligning the segments from different electrodes to a common timeline, accounting for variations in signal timing due to electrode placement and propagation delays. Temporal alignment ensures that the labeled segments from different channels provide a coherent and integrated view of the cardiac activity. Once the segments are aligned and classified, processor 104 may generate the labeled time series segments 132 by annotating each segment with its corresponding label. These labels include detailed information about the identified cardiac events, such as “P-wave,” “QRS complex,” “T-wave,” “atrial fibrillation,” and other relevant classifications. Additionally, each labeled segment includes metadata such as time stamps, duration, and the specific attributes that were used in the classification process.

With continued reference to FIG. 1, a labeled time series segment 132 may include classifying a time series label 136 to each segment attributes 128. As used in current disclosure, a “time series label” is a descriptive annotations assigned to the segment based on its attributes and identified characteristics. This label may be derived from the analysis of various attributes of the segment, such as amplitude, frequency, duration, and morphology, which are indicative of particular physiological or temporal phenomena within the data. A time series label may provide a concise and meaningful description of the time series segment 120, facilitating the interpretation and analysis of the time series data. In the context of cardiac signals, time series labels 136 may be used to identify different phases of the cardiac cycle and for diagnosing potential abnormalities. For example, in the analysis of electrocardiogram (ECG) data 116, A time series label “P-wave” might be assigned to a time series segment 120 representing atrial depolarization, characterized by a small, smooth upward deflection. A time series label “QRS complex” could denote the segment representing ventricular depolarization, identified by its sharp, rapid sequence of deflections. A time series label “T-wave” would indicate ventricular repolarization, marked by a broad, smooth waveform following the QRS complex. Time series labels 136 may be determined through a combination of signal processing and pattern recognition techniques. Processor 104 may employ algorithms that analyze the signal attributes to classify each segment accurately. For instance, the model might use criteria such as the peak amplitude, duration, and shape of the waveform to differentiate between the P-wave, QRS complex, and T-wave. In addition to basic cardiac cycle labels, time series labels 136 may also indicate more complex or pathological events. Labels like “atrial fibrillation” or “ventricular tachycardia” might be used to identify segments where the signal shows irregular rhythms or abnormally high heart rates, respectively. These labels may be crucial for diagnosing and monitoring cardiac conditions, guiding treatment decisions, and conducting further clinical research.

With continued reference to FIG. 1, a time series label 136 may include a diagnostic label. As used in the current disclosure, a “diagnostic label” refers to a term or set of terms assigned to a specific segment of biomedical data (such as time series data from cardiac monitoring devices) to identify and categorize clinical phenomena. These labels may be used to diagnose diseases, guide treatment plans, and monitor patient health over time. The time series label 136 may be a type of diagnostic label that is assigned to segments of data based on their defining characteristics and their clinical significance within a patient's health records. A diagnostic label may be derived from analyzing attributes such as amplitude, frequency, duration, and morphology of the cardiac signals. For example, an algorithm might analyze an ECG waveform to identify a segment with a small, smooth upward deflection as a “P-wave,” indicative of label of “atrial depolarization.” Similarly, a sharp and rapid sequence of deflections would be classified as a “QRS complex,” an be labeled as “ventricular depolarization,” and a broad, smooth waveform following the QRS complex would be labeled as a “T-wave,” denoting ventricular repolarization. Beyond basic cardiac cycle phases, time series labels 136 or diagnostic labels can indicate more complex or pathological conditions. Labels like “atrial fibrillation” (AF) or “ventricular tachycardia” identify segments where the signal demonstrates irregular rhythms or abnormally high heart rates, respectively. Other diagnostic labels within the cardiac domain might include “sinus rhythm” (SINUS) for regular heartbeat patterns, “atrial flutter” (AFL) for rapid contractions of the atria, or “isolated potential” (ISO_POT) for spontaneous depolarization events that may indicate electrical isolation of a heart region. The process of assigning these labels typically involves machine learning models that have been trained on vast datasets of annotated cardiac signals. These models may include but are not limited to convolutional neural networks (CNNs), support vector machines (SVMs), and recurrent neural networks (RNNs). They are capable of recognizing subtle patterns that may not be apparent to the human eye, thereby enhancing the diagnostic process.

With continued reference to FIG. 1, a non-exhaustive list of diagnostic labels may include: sinus rhythm (SINUS), atrial fibrillation (AF), atrial flutter (AFL), pacing from coronary sinus (CPACE), pacing from right atrium (RAPACE), pacing from left atrium (LAPACE), pacing from the ventricle (VPACE), entrainment (ENTRAIN), isolated potential (ISO_POT), reconnection of the pulmonary vein (RECONNECT), right inferior pulmonary vein (RIPV), right superior pulmonary vein (RSPV), left inferior pulmonary vein (LIPV), left superior pulmonary vein (LSPV), left common pulmonary vein (LCPV), posterior wall (POST_WALL), anterior wall (ANT_WALL), left atrial appendage (LAA), stump of LA appendage (LAA_STUMP), left atrium floor (LA_FLOOR), low lateral (LOW_LAT), before ablation (PRE_RFA or PRE_CBA), and after ablation (POST_RFA or POST_CBA). It may be noted, ENTRAIN may be observed when a catheter-delivered pacing impulse captures a local structure or part of the heart, such as in the context of a re-entrant tachycardia. Moreover, isolated potential (ISO_POT) may be captured when spontaneous depolarization occurs indicative of electrical isolation of a part of the heart or structure in which it is observed. In certain cases, the timing of an electrogram in a given part of the heart or structure confirms that it is not part of a re-entrant circuit, such electrogram is referred to as OUT. Further, RECONNECT may be observed presumably after ablation. However, in redo cases, some of the veins that may be labelled are reconnections before the ablations in that particular case begin.

Witch continued reference to FIG. 1, a diagnostic label may be used to identify one or more conditions that are present within the time series segment 120. For example, time series labels such as “sinus rhythm” (SINUS), “atrial fibrillation” (AF), or “ventricular tachycardia” might be used to specify segments depicting regular heartbeats, irregular rhythms, or rapid heart rates, respectively. Processor 104 may classify each segment, employing criteria like peak amplitude, waveform duration, and shape to distinguish between different cardiac events. These labels are crucial for diagnosing and monitoring various cardiac conditions, aiding in treatment decisions and further clinical research.

With continued reference to FIG. 1, the time series label 136 or diagnostic labels may be created based on protocols for collecting data during ablation procedures. The time series collection may occur at different stages, such as pre-ablation, during ablation, and post-ablation. Processor 104 may be configured to establish protocols for collecting ablation-related data from patients. One such protocol involves identifying each mapped structure (or specific part of the heart) recorded by the operator during a clinical procedure. This identification may be based on the procedure log and other associated parameters. A time stamp feature can be used to extract the relevant ablation data at predefined time instances that correspond to specific parameters. For example, a protocol may specify starting data extraction at the parameter “LSPV” at 2:31 PM and ending at “LIPV” at 2:32 PM. Each part of the intracardiac electrogram is then labeled according to the parameters entered by the operator during the procedure. Additionally, other protocols for organizing and naming the electrogram files can be set by the system. Each file name follows a structured format: case number_parameter. The parameter includes the structure or object, rhythm, location, and phase. This standardized file naming convention helps ensure consistency and clarity in data management and retrieval.

With continued reference to FIG. 1, time series labels 136 may be used to train and validate machine learning models. By providing accurate and detailed annotations of the data, these labels enable the models to learn how to recognize and classify different types of events within the time series data. This process improves the model's accuracy and reliability in real-world applications.

With continued reference to FIG. 1, time series labels 136 can originate from various sources, ensuring that the annotated data is accurate and relevant for clinical analysis and machine learning applications. In one embodiment, time series labels 136 may be retrieved from a database of pre-annotated datasets, where each segment has been previously labeled by experts and stored for use in training and validating machine learning models. Alternatively, these labels can be dynamically generated by processor 104, which employs advanced pattern recognition algorithms and machine learning models trained on extensive datasets to classify and label each segment in real-time based on its identified attributes. Additionally, time series labels 136 can be created by medical professionals who manually review the time series data, leveraging their expertise to identify and annotate key events such as P-waves, QRS complexes, T-waves, and pathological conditions like atrial fibrillation. This manual annotation process may provide a high level of accuracy and reliability, especially in complex or ambiguous cases where automated systems may require validation or correction. These diverse sources of time series labels ensure a robust and comprehensive approach to data annotation, supporting effective cardiac diagnosis, monitoring, and treatment.

With continued reference to FIG. 1, processor 104 may classify each time series segment 120 of the plurality of time series segments to at least one time series label 136 as a function of the one or more segment attributes 128. As used in the current disclosure, a “time series classifier” is a classifier that is configured to classify a time series segment 120 to at least one time series label 136. Time series classifier 140 may be consistent with the classifier described below in FIG. 2. Inputs to the time series classifier 140 may include time series data 108, IEGM data 112, ECG data 116, time series segment 120, segment attributes 128, examples of pairs of time series segments 120 and time series labels 136, and the like. Outputs to the time series classifier 140 may include pairs of time series segments 120 and time series labels 136 tailored to each segment attribute 128. Time series training data may include a plurality of data entries containing a plurality of inputs that are correlated to a plurality of outputs for training a processor by a machine-learning process. In an embodiment, time series training data may include a plurality of segment attributes 128 correlated to examples of pairs of time series segments 120 and time series labels 136. Time series training data may be received from database 600. Time series training data may be received from users e.g., experts. Such time series training data may be manually labeled; as a non-limiting example, time series training data may be received from a labeling module e.g., a labeling tool as described in further detail below with reference to FIG. 2. Time series training data may contain information about time series data 108, IEGM data 112, ECG data 116, time series segment 120, segment attributes 128, examples of pairs of time series segments 120 and time series labels 136, and the like. In an embodiment, time series training data may be iteratively updated as a function of the input and output results of past time series classifier 140 or any other classifier mentioned throughout this disclosure. The classifier may use, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifier, and the like.

With continued reference to FIG. 1, a time series classifier 140 may be designed to analyze and classify each time series segment 120 of the plurality of time series segments, assigning at least one time series label 136 based on the segment's attributes 128. The classifier may employ various techniques from machine learning and signal processing to accurately determine the category or type of each segment within the time series data. A time series classifier 140 may receive as an input relevant segment attributes 128 from each segment 120. These attributes can include fundamental features such as amplitude, duration, frequency, and morphology, as well as more complex features like the slope, area under the curve, and temporal intervals. The classifier then uses these attributes as input features for its classification process. In an embodiment, the time series classifier 140 may employ pattern recognition techniques to identify characteristic patterns within the segment. For instance, it may recognize the shape and timing of the P-wave, QRS complex, and T-wave in ECG data, or detect irregular rhythms indicative of conditions like atrial fibrillation. The classifier may be trained using a large dataset of annotated time series data. This training dataset includes labeled examples of various cardiac events, enabling the classifier to learn the distinguishing features of each type of event. Machine learning algorithms such as convolutional neural networks (CNNs), recurrent neural networks (RNNs), or support vector machines (SVMs) are commonly used for this purpose. During the classification phase, the classifier may apply the learned model to new, unlabeled time series segments. By analyzing the extracted attributes and comparing them to the patterns learned during training, the classifier assigns the appropriate label to each segment. For example, a segment with a rapid succession of high-amplitude peaks and troughs might be labeled as a “QRS complex,” while a segment with a smooth, rounded waveform might be labeled as a “T-wave”.

With continued reference to FIG. 1, machine learning may be used to improve the function of apparatus 100. This may include identifying patterns within the segment attributes 128 that lead to changes in the capabilities of the time series classifier 140. By analyzing vast amounts of data related to segment attributes 128, machine learning algorithms can identify patterns, correlations, and dependencies that contribute to the generation of the time series classifier 140. These algorithms can extract valuable insights from various sources, including text, document, EHRs, medical records, medical tests, time series data 108, and the like. By applying machine learning techniques, the software can pair time series segments 120 and time series labels 136 extremely accurately and quickly. Machine learning models may enable the software to learn from past collaborative experiences of the entities and iteratively improve its training data over time.

With continued reference to FIG. 1, processor 104 may be configured to update the time series training data of the time series classifier 140 using user inputs. A time series classifier 140 may use user input to update its training data, thereby improving its performance, speed, and accuracy. In embodiments, the time series classifier 140 may be iteratively updated using input and output results of past iterations of the time series classifiers 140. The time series classifier 140 may then be iteratively retrained using the updated time series training data. For instance, and without limitation, time series classifier 140 may be trained using a first training data from, for example, and without limitation, training data from a user input or database. The time series classifier 140 may then be updated by using previous inputs and outputs from the time series classifier 140 as second set of training data to then retrain a newer iteration of time series classifier 140. This process of updating the time series classifier 140 and its associated training data may be continuously done to create subsequent time series classifiers 140 to improve the speed and accuracy of the time series classifier 140. When users interact with the software, their actions, preferences, and feedback provide valuable information that can be used to refine and enhance the model. This user input is collected and incorporated into the training data, allowing the machine learning model to learn from real-world interactions and adapt its predictions accordingly. By continually incorporating user input, the model becomes more responsive to user needs and preferences, capturing evolving trends and patterns. This iterative process of updating the training data with user input enables the machine learning model to deliver more personalized and relevant results, ultimately enhancing the overall user experience. The discussion within this paragraph may apply to both the time series classifier 140 and any other machine-learning model/classifier discussed herein.

Incorporating the user feedback may include updating the training data by removing or adding correlations of user data to a path or resources as indicated by the feedback. Any machine-learning model as described herein may have the training data updated based on such feedback or data gathered using any method described herein. For example, when correlations in training data are based on outdated information, a web crawler may update such correlations based on more recent resources and information.

With continued reference to FIG. 1, processor 104 may use user feedback to train the machine-learning models and/or classifiers described above. For example, machine-learning models and/or classifiers may be trained using past inputs and outputs of the machine-learning model. In some embodiments, if user feedback indicates that an output of machine-learning models and/or classifiers was “unfavorable,” then that output and the corresponding input may be removed from training data used to train machine-learning models and/or classifiers, and/or may be replaced with a value entered by, e.g., another value that represents an ideal output given the input the machine learning model originally received, permitting use in retraining, and adding to training data; in either case, machine-learning models may be retrained with modified training data as described in further detail below. In some embodiments, training data of classifier may include user feedback.

With continued reference to FIG. 1, in some embodiments, an accuracy score may be calculated for the machine-learning model and/or classifier using user feedback. For the purposes of this disclosure, “accuracy score,” is a numerical value concerning the accuracy of a machine-learning model. For example, the accuracy/quality of the output time series classifier 140 may be averaged to determine an accuracy score. In some embodiments, an accuracy score may be determined for pairing of entities. Accuracy score or another score as described above may indicate a degree of retraining needed for a machine-learning model and/or classifier. Processor 104 may perform a larger number of retraining cycles for a higher number (or lower number, depending on a numerical interpretation used), and/or may collect more training data for such retraining. The discussion within this paragraph and the paragraphs preceding this paragraph may apply to both the time series classifier 140 and/or any other machine-learning model/classifier mentioned herein.

With continued reference to FIG. 1, processor 104 may be configured to generate a time series report 144 as a function of the labeled time series segment. As used in the current disclosure, a “time series report” refers to a document that integrates and analyzes sequential data points over time derived from specific medical diagnostic tests such as Intracardiac Electrogram (IEGM) reports and Electrocardiogram (ECG) reports, to monitor and evaluate cardiac functions and anomalies. For instance, an IEGM report might focus on the detailed temporal sequence of electrical signals within the heart's chambers, identifying specific patterns or anomalies like premature ventricular contractions or atrial fibrillation. This report would use the labeled segments of IEGM data to correlate these events with clinical symptoms or potential arrhythmias, offering a chronological insight into the electrical activity and its implications for cardiac health. Similarly, an ECG report generated as a function of a labeled time series segment might provide a sequential analysis of the heart's electrical activity, detailing the timing and morphology of waves and intervals like the P wave, QRS complex, and T wave. This could include identifying trends or changes in the heart rate variability, the presence of late potentials, or the progression of conditions such as ischemia or hypertrophy over time. Each report would systematically detail these findings, offering a comprehensive view of the heart's electrical behavior through successive measurements. In an embodiment, these time series reports might also compare current data against historical data, highlighting trends or deviations that are crucial for diagnosing progression or improvement of cardiac conditions. They may suggest specific therapeutic interventions, further testing, or lifestyle modifications based on the analyzed data. This comparative and temporal analysis not only provides a detailed snapshot of current cardiac function but also tracks changes over time, aiding in the management of chronic cardiac conditions. Each section of the report would be meticulously formatted to ensure clarity, often supplemented with graphical representations of the time series data to enhance comprehension, and provide a visual track of the heart's electrical activity over time.

With continued reference to FIG. 1, generating a time series report for cardiac functions may be done using a machine learning model. Labeled time series segments may be fed into a supervised machine learning model designed to recognize and categorize patterns in cardiac electrical activity. The model may be trained on labeled datasets to identify specific cardiac events, such as arrhythmias, conduction blocks, or myocardial infarction indicators, learning to correlate these events with clinical outcomes. Once trained, the model may analyze new time series data, automatically classifying segments based on learned patterns and generating detailed reports. These reports may include a narrative of the heart's electrical activity, changes over time, and potential clinical implications, formatted for clarity and supplemented with visual graphs derived from the analyzed data. Additionally, the model could utilize historical data comparisons to highlight trends or deviations, aiding in diagnosis and the monitoring of treatment efficacy. This approach leverages the predictive power of machine learning to provide precise, automated, and highly informative cardiac health assessments.

Still referring to FIG. 1, processor 104 may be configured to display the at least one labeled time series segment using a display device 148. As used in the current disclosure, a “display device” is a device that is used to display a plurality of data and other digital content. A display device 148 may include a user interface. A “user interface,” as used herein, is a means by which a user and a computer system interact; for example through the use of input devices and software. A user interface may include a graphical user interface (GUI), command line interface (CLI), menu-driven user interface, touch user interface, voice user interface (VUI), form-based user interface, any combination thereof, and the like. A user interface may include a smartphone, smart tablet, desktop, or laptop operated by the user. In an embodiment, the user interface may include a graphical user interface. A “graphical user interface (GUI),” as used herein, is a graphical form of user interface that allows users to interact with electronic devices. In some embodiments, GUI may include icons, menus, other visual indicators, or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pulldown menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access. Information contained in user interface may be directly influenced using graphical control elements such as widgets. A “widget,” as used herein, is a user control element that allows a user to control and change the appearance of elements in the user interface. In this context a widget may refer to a generic GUI element such as a check box, button, or scroll bar to an instance of that element, or to a customized collection of such elements used for a specific function or application (such as a dialog box for users to customize their computer screen appearances). User interface controls may include software components that a user interacts with through direct manipulation to read or edit information displayed through user interface. Widgets may be used to display lists of related items, navigate the system using links, tabs, and manipulate data using check boxes, radio boxes, and the like.

Referring now to FIG. 2, a flow diagram of an exemplary implementation of a machine learning pipeline 200 for generating a label (e.g., time-series label) for labeling time-series data according to one embodiment is illustrated. In some cases, time-series data may include an intracardiac electrogram. The machine learning pipeline may include data collection, data validation, standardization, storage labeling, and data creation. In some embodiments, such machine learning pipeline may be used for training data generation. Time series training data may be generated by standardizing each one of the examples of segment attributes into a canonical data format, verifying, for each one of the standardized examples of segment attributes, against a set of pre-defined configuration settings, and annotating, at a labeling module, the verified examples of segment attributes with examples of time series labels to generate time series training data.

With continued reference to FIG. 2, as a non-limiting example, at 202, de-identified case data from institutions may be received. This de-identified cases data may correspond to an un-identified and unlabeled intracardiac electrogram. At 204, apparatus may be configured to perform data validation on the de-identified case data. Additionally, or alternatively, the de-identified data may be formatted based on scripts; for instance, and without limitation, the scripts may load vital configuration settings from a configuration file. Configuration settings play a pivotal role in shaping the data preparation workflow. As a non-limiting example, configuration settings may include one or more key configuration parameters including, but is not limited to, a list of leads relevant to the de-identified case data, an output directory where processed data will be stored, a label directory for label data, a list of test cases for which labels need to be separated, a CSV file that contains metadata (e.g., anatomy, ablation type, and the like) about the deidentified case data, a list of valid lengths for label data, and the like. Further, each file name may be structured in the following manner: case number parameter, where (parameter) is formatted in the following structure: rhythm_location_phase.

With continued reference to FIG. 2, continuing the non-limiting example, at 206, the validated de-identified case data may be transmitted to a cloud platform, for example, an AWS cloud. At 208, each of the case data may be converted to a canonical data format based on the scripts. As described herein, a “canonical data format” is a standardized consistent structure used to represent data across one or more processing steps as described herein. In an embodiment, data in canonical data format may be easily interpretable, comparable, and/or integrable regardless of its source or initial structured. At 210, a converted data validation may be performed for formatting and integrity check. Additionally, or alternatively, the converted canonical data validation on each converted case data may be performed based on an identifier associated with leads, a size, and the like specified in the script. Further, after the canonical data validation, each converted case data may be transmitted to the cloud platform. At 212, each converted case data may be input to a labeling module. As described herein, a “labeling module” is a program configured to assign labels to time series segments. As a non-limiting example, labeling module may include a labeling tool implemented to facilitate expert annotation. In some cases, human experts may review and adjust time series labels when inaccurate time series labels are detected. Additionally, or alternatively, the labeling module, such as labeling tool may receive each converted case data from the cloud platform. Further, at 214, clinicians/experts provide one or more labels corresponding to each converted case data. At 216, the policy or protocols for the one or more labels may be generated. At 218, training/development/testing data is performed based on the policy and each converted each case data.

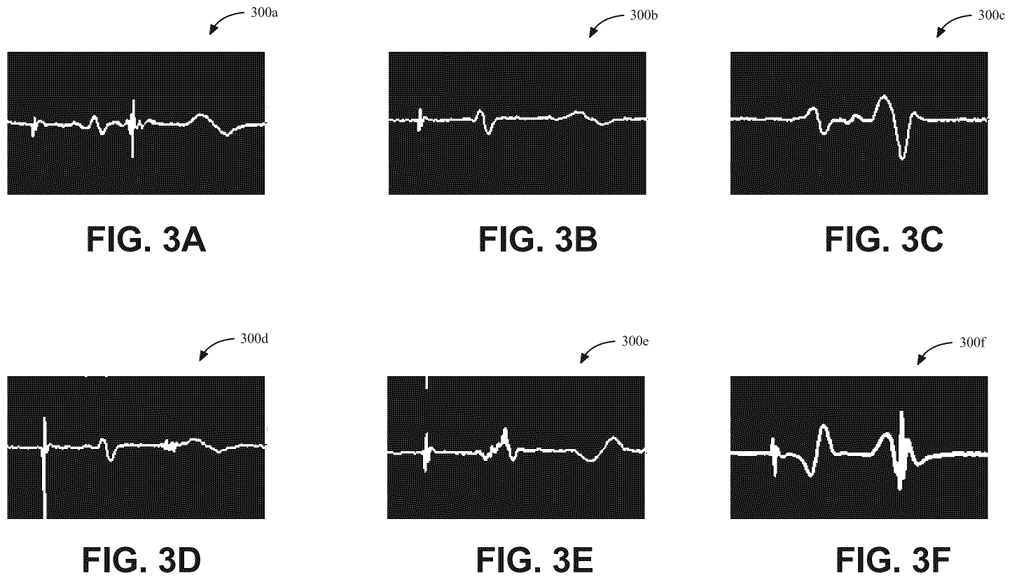

Now referring to FIGS. 3A-F, exemplary embodiments of labeling strategies for generating labels corresponding to a plurality of intracardiac electrogram 300a-f is illustrated. Apparatus as described herein may include a system for pre-processing de-identified case data for aiding generation of a set of time-series labels for an intracardiac electrogram. As a non-limiting example, such system may include time series classifier configured to label an intracardiac electrogram by labeling multiple segments of the intracardiac electrogram for object detection. Further, time series classifier may be configured to label the multiple segments of the intracardiac electrogram to diagnose and treat the cardiac abnormalities associated with various parts or structures of the heart.

With continued reference to FIGS. 3A-F, intracardiac electrograms 300a-f may illustrate various PVP signals. In particular, Pulmonary vein (PV) potentials are invariably recordable at the PV ostia in patients with atrial fibrillation (AF) and delayed conduction around the PV ostia may play a role in the initiation and maintenance of AF. Atrial fibrillation (AF) is currently the most challenging arrhythmia in clinical practice. There has been clear anatomical evidence that myocardial sleeves extend into the pulmonary veins (PVs) and are capable of conducting electrical impulses and generating ectopic beats as triggers for initiation of AF. PVs may also play a role in the maintenance of AF, due to the complicated architecture of the myocardial sleeves surrounding the ostium of the PVs and their heterogeneity in conduction properties and refractoriness. In the clinical setting, it has become clear that typical PV potentials are almost invariably recordable at the PV ostium in patients with AF and ablation around the PV ostium leads to curative results. These findings suggest that electrical activities around the ostia of PVs are of clinical importance.

Referring to FIG. 3A, an example intracardiac electrogram 300a that is labelled corresponding to PVP is shown. The label for the intracardiac electrogram 300a for PVP corresponds to “yes”, i.e., “presence of PVP signal” when appropriate activation sequence is observed, clear near-field electrogram is observed, no fusion between PVP and far-field EGM is observed, and isoelectric intervals are visible between PVP and far-field EGMs (or at least return to baseline).

Referring to FIG. 3B, an example intracardiac electrogram 300b that is labelled corresponding to PVP is shown. The label for the intracardiac electrogram 300b for PVP corresponds to “no”, i.e., “absence of PVP signal” when no electrogram is visible when PVP is expected.

Referring to FIGS. 3C-3F, an example intracardiac electrogram that is labelled corresponding to PVP is shown. The label for PVP corresponds to “maybe”, i.e., “unsure of presence or absence of PVP signal”. In FIG. 3C, the intracardiac electrogram 300c is labelled “maybe” as the intracardiac electrogram 300c has a timing consistent with PVP, but with a far-field waveform. Further, in FIG. 3D, the intracardiac electrogram 300d is labelled “maybe” as the intracardiac electrogram 300d has a timing consistent with PVP with near-field waveform, but an amplitude of the waveform is less than waveform adjoining far-field signal, i.e., lacking a dominant peak. In FIG. 3E, the intracardiac electrogram 300c is labelled “maybe” as the intracardiac electrogram 300e is a near-field electrogram consistent with PVP present but fused with far-field atrial signal. Further, in FIG. 3F, the intracardiac electrogram 300f is labelled “maybe” as the intracardiac electrogram 300f is a near-field electrogram consistent with PVP present but fused with far-field ventricular signal.

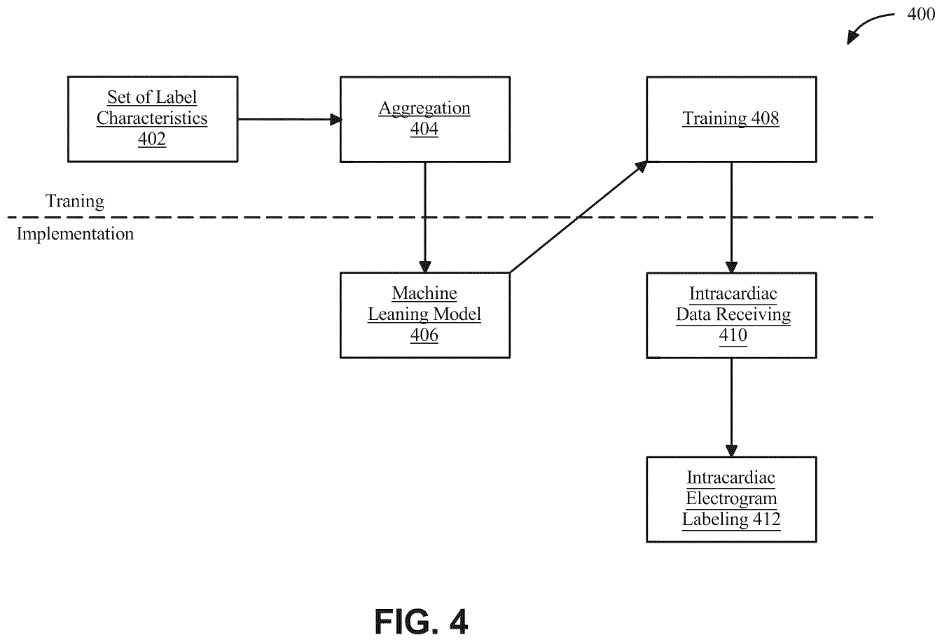

Now referring to FIG. 4, a block diagram of an exemplary embodiment 400 of training a machine learning model 406 used for labeling of the time-series data is illustrated. ML model 406 may include any ML model as described herein such as, without limitation, time series classifier may be first trained and then implemented as described above with reference to FIG. 1. During model training, a set of label characteristics 402 may be determined or identified from various training data, such as, without limitation, example time series data including example intracardiac electrograms or segments of training intracardiac electrograms. The training intracardiac electrograms or their segments may be labelled based on a set of time-series labels. Further, set of label characteristics 402 for different labels in the set of labels are aggregated 404 to learn to label an intracardiac electrogram. Further, after aggregation 404, set of label characteristics 402 may be provided to ML model 406 for the training 408 of ML model 406.

With continued reference to FIG. 4, in the implementation phase, data 410 may be received. Processor may be configured to perform labeling 412 by correlating one or more segments of the received intracardiac electrogram with set of label characteristics 402 for the set of labels. Additionally, or alternatively, ML model 406 may be re-trained using one or more re-training techniques. The one or more re-training techniques may include, but are not limited to, batch re-training techniques, active learning re-training techniques, incremental learning re-training techniques, transfer learning re-training techniques, reinforcement learning policies re-training techniques, ensemble method re-training techniques, data augmentation re-training techniques, adaptive learning rates re-training techniques, and the like. Accordingly, a user interface may be provided to display the labeled one or more segments of the received intracardiac electrogram.

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example examples of segment attributes correlated to examples of time series labels.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data according to a given condition or diagnosis. For example, training data may be classified according to the presence of a condition like atrial fibrillation.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Still referring to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 556 pixels, however a desired number of pixels may be 128. Processor may down-sample the high pixel count image to convert the 556 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include segment attributes and examples of time series labels as described above as inputs, labeled time series segments as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized, or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

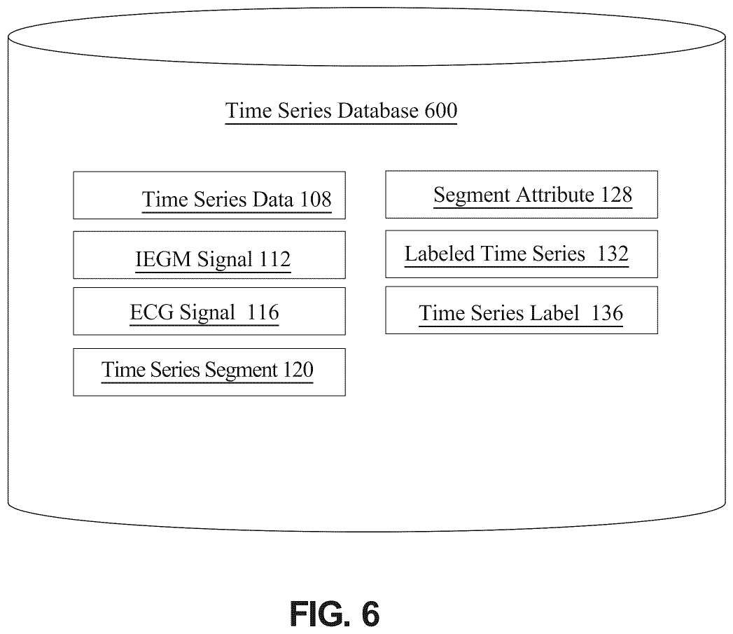

Now referring to FIG. 6, an exemplary time series database 600 is illustrated by way of block diagram. In an embodiment, any past or present versions of any data disclosed herein may be stored within the time series database 600 including but not limited to: time series data 108, IEGM data 112, ECG data 116, time series segment 120, segment attributes 128, labeled time series segment 132, time series labels 136, and the like. Processor 104 may be communicatively connected with time series database 600. For example, in some cases, database 600 may be local to processor 104. Alternatively or additionally, in some cases, database 600 may be remote to processor 104 and communicative with processor 104 by way of one or more networks. Network may include, but not limited to, a cloud network, a mesh network, or the like. By way of example, a “cloud-based” system, as that term is used herein, can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor 104 connects directly, dynamically, and non-hierarchically to as many other computing devices as possible. A “network topology” as used in this disclosure is an arrangement of elements of a communication network. time series database 600 may be implemented, without limitation, as a relational database, a key-value retrieval database such as a NOSQL database, or any other format or structure for use as a database that a person skilled in the art would recognize as suitable upon review of the entirety of this disclosure. time series database 600 may alternatively or additionally be implemented using a distributed data storage protocol and/or data structure, such as a distributed hash table or the like. time series database 600 may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

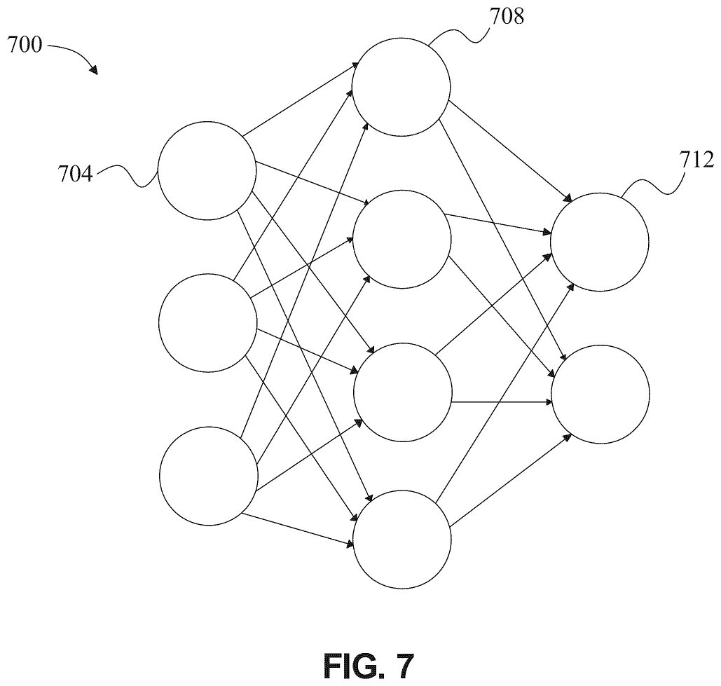

Referring now to FIG. 7, an exemplary embodiment of neural network 700 is illustrated. A neural network 700, also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 704, one or more intermediate layers 708, and an output layer of nodes 712. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

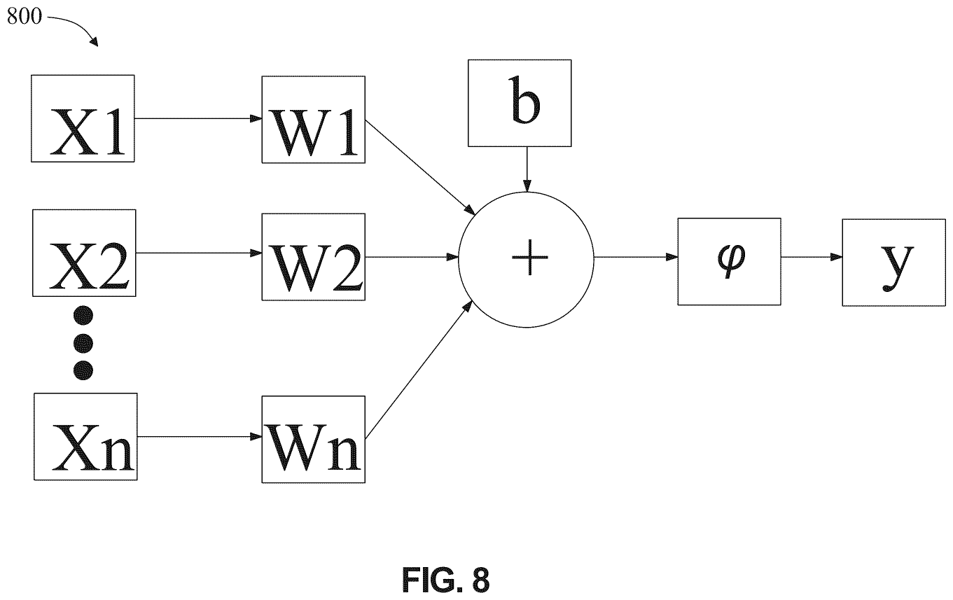

Referring now to FIG. 8, an exemplary embodiment 800 of a node of a neural network is illustrated. A node may include, without limitation, a plurality of inputs xᵢ that may receive numerical values from inputs to a neural network containing the node and/or from other nodes. Node may perform a weighted sum of inputs using weights w; that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights w; may be determined by training a neural network using training data, which may be performed using any suitable process as described above.

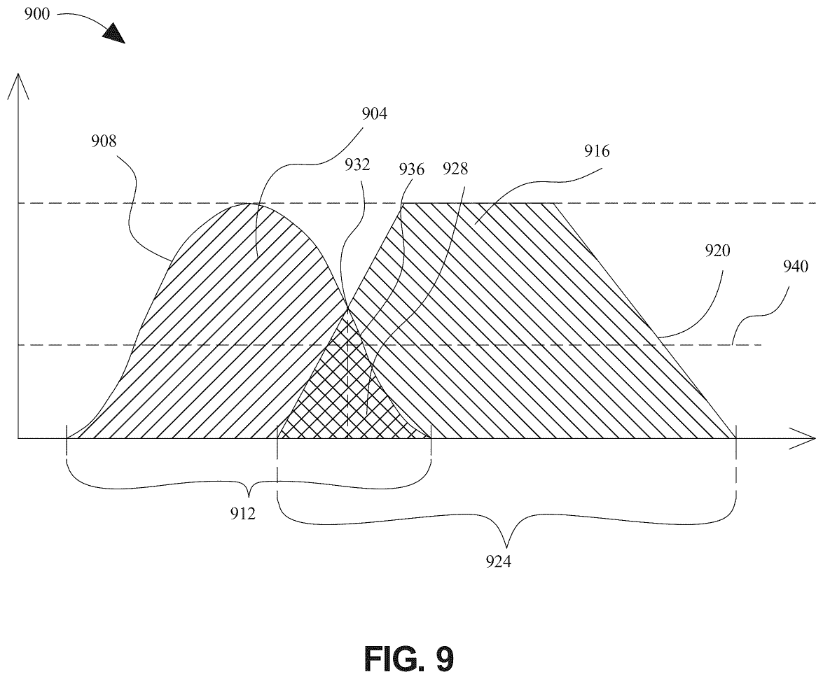

Now referring to FIG. 9, an exemplary embodiment of fuzzy set comparison 900 is illustrated. In a non-limiting embodiment, the fuzzy set comparison. In a non-limiting embodiment, fuzzy set comparison 900 may be consistent with fuzzy set comparison in FIG. 1. In another non-limiting the fuzzy set comparison 900 may be consistent with the name/version matching as described herein. For example and without limitation, the parameters, weights, and/or coefficients of the membership functions may be tuned using any machine-learning methods for the name/version matching as described herein. In another non-limiting embodiment, the fuzzy set may represent a segment attributes 128 and a time series labels 136 from FIG. 1.

Alternatively or additionally, and still referring to FIG. 9, fuzzy set comparison 900 may be generated as a function of determining the data compatibility threshold. The compatibility threshold may be determined by a computing device. In some embodiments, a computing device may use a logic comparison program, such as, but not limited to, a fuzzy logic model to determine the compatibility threshold and/or version authenticator. Each such compatibility threshold may be represented as a value for a posting variable representing the compatibility threshold, or in other words a fuzzy set as described above that corresponds to a degree of compatibility and/or allowability as calculated using any statistical, machine-learning, or other method that may occur to a person skilled in the art upon reviewing the entirety of this disclosure. In some embodiments, determining the compatibility threshold and/or version authenticator may include using a linear regression model. A linear regression model may include a machine learning model. A linear regression model may map statistics such as, but not limited to, frequency of the same range of version numbers, and the like, to the compatibility threshold and/or version authenticator. In some embodiments, determining the compatibility threshold of any posting may include using a classification model. A classification model may be configured to input collected data and cluster data to a centroid based on, but not limited to, frequency of appearance of the range of versioning numbers, linguistic indicators of compatibility and/or allowability, and the like. Centroids may include scores assigned to them such that the compatibility threshold may each be assigned a score. In some embodiments, a classification model may include a K-means clustering model. In some embodiments, a classification model may include a particle swarm optimization model. In some embodiments, determining a compatibility threshold may include using a fuzzy inference engine. A fuzzy inference engine may be configured to map one or more compatibility threshold using fuzzy logic. In some embodiments, a plurality of computing devices may be arranged by a logic comparison program into compatibility arrangements. A “compatibility arrangement” as used in this disclosure is any grouping of objects and/or data based on skill level and/or output score. Membership function coefficients and/or constants as described above may be tuned according to classification and/or clustering algorithms. For instance, and without limitation, a clustering algorithm may determine a Gaussian or other distribution of questions about a centroid corresponding to a given compatibility threshold and/or version authenticator, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

Still referring to FIG. 9, inference engine may be implemented according to input segment attributes 128 and time series labels 136. For instance, an acceptance variable may represent a first measurable value pertaining to the classification of segment attributes 128 to time series labels 136. Continuing the example, an output variable may represent labeled time series segment 132 associated with the user. In an embodiment, segment attributes 128 and/or time series labels 136 may be represented by their own fuzzy set. In other embodiments, the classification of the data into labeled time series segment 132 may be represented as a function of the intersection two fuzzy sets as shown in FIG. 9, An inference engine may combine rules, such as any semantic versioning, semantic language, version ranges, and the like thereof. The degree to which a given input function membership matches a given rule may be determined by a triangular norm or “T-norm” of the rule or output function with the input function, such as min (a, b), product of a and b, drastic product of a and b, Hamacher product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “⊥” such as max (a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Area defuzzification, or the like. Alternatively or additionally, output rules may be replaced with functions according to the Takagi-Sugeno-King (TSK) fuzzy model.

A first fuzzy set 904 may be represented, without limitation, according to a first membership function 908 representing a probability that an input falling on a first range of values 912 is a member of the first fuzzy set 904, where the first membership function 908 has values on a range of probabilities such as without limitation the interval [0,1], and an area beneath the first membership function 908 may represent a set of values within first fuzzy set 904. Although first range of values 912 is illustrated for clarity in this exemplary depiction as a range on a single number line or axis, first range of values 912 may be defined on two or more dimensions, representing, for instance, a Cartesian product between a plurality of ranges, curves, axes, spaces, dimensions, or the like. First membership function 908 may include any suitable function mapping first range of values 912 to a probability interval, including without limitation a triangular function defined by two linear elements such as line segments or planes that intersect at or below the top of the probability interval. As a non-limiting example, triangular membership function may be defined as:

$$( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

First fuzzy set 904 may represent any value or combination of values as described above, including any segment attributes 128 and time series labels 136. A second fuzzy set 916, which may represent any value which may be represented by first fuzzy set 904, may be defined by a second membership function 920 on a second range 924; second range 924 may be identical and/or overlap with first range of values 912 and/or may be combined with first range via Cartesian product or the like to generate a mapping permitting evaluation overlap of first fuzzy set 904 and second fuzzy set 916. Where first fuzzy set 904 and second fuzzy set 916 have a region 936 that overlaps, first membership function 908 and second membership function 920 may intersect at a point 932 representing a probability, as defined on probability interval, of a match between first fuzzy set 904 and second fuzzy set 916. Alternatively or additionally, a single value of first and/or second fuzzy set may be located at a region 936 on first range of values 912 and/or second range 924, where a probability of membership may be taken by evaluation of first membership function 908 and/or second membership function 920 at that range point. A probability at 928 and/or 932 may be compared to a threshold 940 to determine whether a positive match is indicated. Threshold 940 may, in a non-limiting example, represent a degree of match between first fuzzy set 904 and second fuzzy set 916, and/or single values therein with each other or with either set, which is sufficient for purposes of the matching process; for instance, the classification into one or more query categories may indicate a sufficient degree of overlap with fuzzy set representing segment attributes 128 and time series labels 136 for combination to occur as described above. Each threshold may be established by one or more user inputs. Alternatively or additionally, each threshold may be tuned by a machine-learning and/or statistical process, for instance and without limitation as described in further detail below.

In an embodiment, a degree of match between fuzzy sets may be used to rank one resource against another. For instance, if both segment attributes 128 and time series labels 136 have fuzzy sets, labeled time series segments 132 may be generated by having a degree of overlap exceeding a predictive threshold, processor 104 may further rank the two resources by ranking a resource having a higher degree of match more highly than a resource having a lower degree of match. Where multiple fuzzy matches are performed, degrees of match for each respective fuzzy set may be computed and aggregated through, for instance, addition, averaging, or the like, to determine an overall degree of match, which may be used to rank resources; selection between two or more matching resources may be performed by selection of a highest-ranking resource, and/or multiple notifications may be presented to a user in order of ranking.

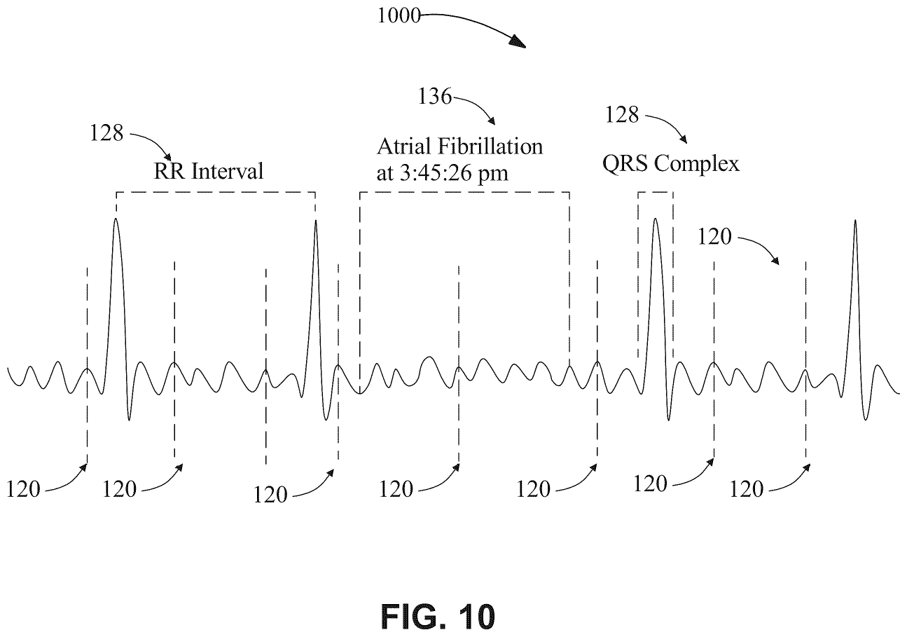

Referring now to FIG. 10, an exemplary embodiment of a labeled time series segment 1000 is depicted. This segment 120 labeled with a diagnostic label reflecting atrial fibrillation (AF). Atrial fibrillation may be characterized by irregular and often rapid heartbeats originating from disorganized electrical signals in the atria. In this labeled segment 1000, the hallmark features of AF are evident: there is an absence of distinct P-waves, which normally represent atrial depolarization, and the baseline appears erratic with continuous, chaotic fibrillatory waves. The QRS complexes, which denote ventricular depolarization, occur at irregular intervals, further indicating the presence of atrial fibrillation. This diagnostic label may highlight the presence of this common and serious arrhythmia, aiding in prompt and accurate diagnosis. This labeled time series segment provides a clear example of how the system identifies and marks significant cardiac events, ensuring that atrial fibrillation is readily recognized for appropriate medical intervention.

With continued reference to FIG. 10, the labeled features of the labeled time series segment 1000 may include several distinct characteristics that are indicative of this arrhythmia. First, processor 104 may identify the absence of distinct P-waves; in a normal ECG, P-waves may represent the orderly atrial depolarization, but in atrial fibrillation, these waves may be replaced by erratic fibrillatory waves. These fibrillatory waves are irregular in amplitude and frequency, creating a chaotic baseline instead of the smooth, predictable pattern seen in normal sinus rhythm. Additionally, the QRS complexes, which represent ventricular depolarization, occur at irregular intervals. This irregularity is due to the disorganized electrical activity in the atria, which leads to an unpredictable conduction of impulses to the ventricles. The variability in the R-R intervals (the time between successive R-waves) is a key diagnostic feature of AF. The overall morphology of the labeled segment shows a lack of regularity and consistency, contrasting sharply with the regular, periodic structure of a normal ECG. The segment may also show variations in the amplitude of the QRS complexes due to inconsistent atrial input to the ventricles.

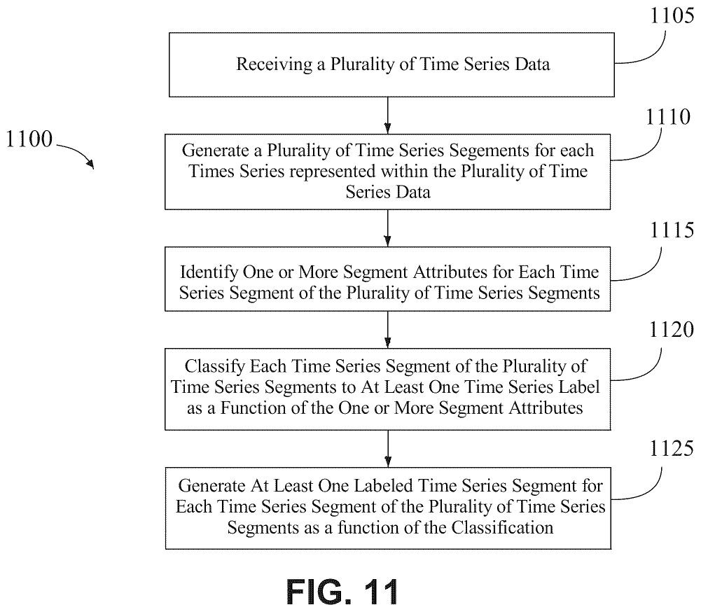

Referring now to FIG. 11, a flow diagram of an exemplary method 1100 for labeling a plurality of time series data is illustrated. At step 1105, method 1100 includes receiving a plurality of time series data. This may be implemented as described and with reference to FIGS. 1-10. In an embodiment, the plurality of time series data may include a plurality of intracardiac electrogram (IEGM) signals or ECG signals. In an embodiment, receiving the plurality of intracardiac electrograms signals may include receiving the plurality of intracardiac electrograms signals from a catheter. In an additional embodiment, receiving the plurality of time series data comprises receiving the plurality of time series data from an application program interface.

Still referring to FIG. 11, at step 1110, method 1100 includes generating, using the at least a processor, a plurality of time series segments for each time series represented within the plurality of time series data. This may be implemented as described and with reference to FIGS. 1-10. In an embodiment, the method may further include preprocessing, using the at least a processor, the plurality of time series data using a using one or more digital filtering techniques.

Still referring to FIG. 11, at step 1115, method 1100 includes identifying, using the at least a processor, one or more segment attributes for each time series segment of the plurality of time series segments. This may be implemented as described and with reference to FIGS. 1-10. In an embodiment, the method may further include generating, using the at least a processor, an attribute score as a function of the one or more segment attributes

Still referring to FIG. 11, at step 1120, method 1100 includes classifying, using the at least a processor each time series segment of the plurality of time series segments to at least one time series label as a function of the one or more segment attributes. This may be implemented as described and with reference to FIGS. 1-10. In some cases, training the times series classifier may include updating the time series training data as a function of an input and outputs of a previous time series classifier; and retraining the time series classifier using the updated time series training data.

Still referring to FIG. 8, at step 1125, method 1100 includes generating, using the at least a processor, at least one labeled time series segment for each time series segment of the plurality of time series segments as a function of the classification. This may be implemented as described and with reference to FIGS. 1-10. In an embodiment, the method may further include method generating, using the at least a processor, a times series report as a function of the one or more segment attributes. In an additional embodiment, the method may further include performing, using the at least a processor, a temporal alignment of the labeled time series segments.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 9 shows a diagrammatic representation of one embodiment of a computing device in the exemplary form of a computer system 1200 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1200 includes a processor 1204 and a memory 1208 that communicate with each other, and with other components, via a bus 1212. Bus 1212 may include any of several types of bus structures including, but not limited to, a memory bus, a memory controller, a peripheral bus, a local bus, and any combinations thereof, using any of a variety of bus architectures.

Processor 1204 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1204 may be organized according to Von Neumann and/or Harvard architecture as a non-limiting example. Processor 1204 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1208 may include various components (e.g., machine-readable media) including, but not limited to, a random-access memory component, a read only component, and any combinations thereof. In one example, a basic input/output system 1216 (BIOS), including basic routines that help to transfer information between elements within computer system 1200, such as during start-up, may be stored in memory 1208. Memory 1208 may also include (e.g., stored on one or more machine-readable media) instructions (e.g., software) 1220 embodying any one or more of the aspects and/or methodologies of the present disclosure. In another example, memory 1208 may further include any number of program modules including, but not limited to, an operating system, one or more application programs, other program modules, program data, and any combinations thereof.

Computer system 1200 may also include a storage device 1224. Examples of a storage device (e.g., storage device 1224) include, but are not limited to, a hard disk drive, a magnetic disk drive, an optical disc drive in combination with an optical medium, a solid-state memory device, and any combinations thereof. Storage device 1224 may be connected to bus 1212 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1224 (or one or more components thereof) may be removably interfaced with computer system 1200 (e.g., via an external port connector (not shown)). Particularly, storage device 1224 and an associated machine-readable medium 1228 may provide nonvolatile and/or volatile storage of machine-readable instructions, data structures, program modules, and/or other data for computer system 1200. In one example, software 1220 may reside, completely or partially, within machine-readable medium 1228. In another example, software 1220 may reside, completely or partially, within processor 1204.

Computer system 1200 may also include an input device 1232. In one example, a user of computer system 1200 may enter commands and/or other information into computer system 1200 via input device 1232. Examples of an input device 1232 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1232 may be interfaced to bus 1212 via any of a variety of interfaces (not shown) including, but not limited to, a serial interface, a parallel interface, a game port, a USB interface, a FIREWIRE interface, a direct interface to bus 1212, and any combinations thereof. Input device 1232 may include a touch screen interface that may be a part of or separate from display device 1236, discussed further below. Input device 1232 may be utilized as a user selection device for selecting one or more graphical representations in a graphical interface as described above.

A user may also input commands and/or other information to computer system 1200 via storage device 1224 (e.g., a removable disk drive, a flash drive, etc.) and/or network interface device 1240. A network interface device, such as network interface device 1240, may be utilized for connecting computer system 1200 to one or more of a variety of networks, such as network 1244, and one or more remote devices 1248 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1244, may employ a wired and/or a wireless mode of communication. In general, any network topology may be used. Information (e.g., data, software 1220, etc.) may be communicated to and/or from computer system 1200 via network interface device 1240.

Computer system 1200 may further include a video display adapter 1252 for communicating a displayable image to a display device, such as display device 1236. Examples of a display device include, but are not limited to, a liquid crystal display (LCD), a cathode ray tube (CRT), a plasma display, a light emitting diode (LED) display, and any combinations thereof. Display adapter 1252 and display device 1236 may be utilized in combination with processor 1204 to provide graphical representations of aspects of the present disclosure. In addition to a display device, computer system 1200 may include one or more other peripheral output devices including, but not limited to, an audio speaker, a printer, and any combinations thereof. Such peripheral output devices may be connected to bus 1212 via a peripheral interface 1256. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

1. An apparatus for labeling a plurality of time series data, wherein the apparatus comprises:
at least a processor; and
a memory communicatively connected to the at least a processor, wherein the memory contains instructions configuring the at least a processor to:
receive a plurality of time series data;
generate a plurality of time series segments for each time series represented within the plurality of time series data, wherein the at least a processor employs a temporal alignment technique to synchronize the segments across multiple channels of data, including at least recordings from different electrodes, wherein the technique comprises stretching or compressing segments of the time series to output a temporal alignment;
identify one or more segment attributes for each time series segment of the plurality of time series segments;
classify each time series segment of the plurality of time series segments to at least one time series label as a function of the one or more segment attributes, wherein classifying each time series segment of the plurality of time series segments to at least one time series label comprises:
training a time series classifier using time series training data, wherein the time series training data comprises examples of segment attributes correlated to examples of time series labels; and
classifying each time series segment of the plurality of time series segments to at least one time series label using the trained time series classifier; and
generate at least one labeled time series segment for each time series segment of the plurality of time series segments as a function of the classification.

2. The apparatus of claim 1, wherein the plurality of time series data comprises a plurality of intracardiac electrogram (IEGM) signals.

3. The apparatus of claim 2, wherein receiving the plurality of intracardiac electrograms signals comprises receiving the plurality of intracardiac electrograms signals from a catheter.

4. The apparatus of claim 1, wherein the plurality of time series data comprises a plurality of electrocardiogram (ECG) signals.

5. The apparatus of claim 1, wherein training the time series classifier comprises generating the time series training data using a labeling module.

6. The apparatus of claim 5, wherein generating the time series training data comprises:
standardizing each one of the examples of segment attributes into a canonical data format; and
verifying, for each one of the standardized examples of segment attributes, against a set of pre-defined configuration settings; and
annotating, at the labeling module, the verified examples of segment attributes with the examples of time series labels to generate time series training data.

7. The apparatus of claim 1, wherein the memory further instructs the at least a processor to generate an attribute score as a function of the one or more segment attributes.

8. The apparatus of claim 1, wherein the memory further instructs the at least a processor to generate a times series report as a function of the one or more segment attributes.

9. The apparatus of claim 1, wherein training the training a time series classifier comprises:
updating the time series training data as a function of an input and outputs of a previous time series classifier; and
retraining the time series classifier using the updated time series training data.

10. The apparatus of claim 1, wherein the memory further instructs the at least a processor to perform a temporal alignment of the labeled time series segments.

11. A method for labeling a plurality of time series data, wherein the method comprises:
receiving, using at least a processor, a plurality of time series data;
generating, using the at least a processor, a plurality of time series segments for each time series represented within the plurality of time series data, wherein the at least a processor employs a temporal alignment technique to synchronize the segments across multiple channels of data, including at least recordings from different electrodes, wherein the technique comprises stretching or compressing segments of the time series to output a temporal alignment;
identifying, using the at least a processor, one or more segment attributes for each time series segment of the plurality of time series segments;
classifying, using the at least a processor each time series segment of the plurality of time series segments to at least one time series label as a function of the one or more segment attributes, wherein classifying each time series segment of the plurality of time series segments to at least one time series label comprises:
training a time series classifier using time series training data, wherein time series training data comprises examples of segment attributes correlated to examples of time series labels; and
classifying each time series segment of the plurality of time series segments to at least one time series label using the trained time series classifier; and
generating, using the at least a processor, at least one labeled time series segment for each time series segment of the plurality of time series segments as a function of the classification.

12. The method of claim 11, wherein the plurality of time series data comprises a plurality of intracardiac electrogram (IEGM) signals.

13. The method of claim 12, wherein receiving the plurality of intracardiac electrograms signals comprises receiving the plurality of intracardiac electrograms signals from a catheter.

14. The method of claim 11, wherein the plurality of time series data comprises a plurality of electrocardiogram (ECG) signals.

15. The method of claim 11, wherein training the time series classifier comprises generating the time series training data using a labeling module.

16. The method of claim 15, wherein generating the time series training data comprises:
standardizing each one of the examples of segment attributes into a canonical data format; and
verifying, for each one of the standardized examples of segment attributes, against a set of pre-defined configuration settings; and
annotating, at the labeling module, the verified examples of segment attributes with the examples of time series labels to generate time series training data.

17. The method of claim 11, wherein the method further comprises generating, using the at least a processor, an attribute score as a function of the one or more segment attributes.

18. The method of claim 11, wherein the method further comprises generating, using the at least a processor, a times series report as a function of the one or more segment attributes.

19. The method of claim 11, wherein training the training a time series classifier comprises:
updating the time series training data as a function of an input and outputs of a previous time series classifier; and
retraining the time series classifier using the updated time series training data.

20. The method of claim 11, wherein the method further comprises performing, using the at least a processor, a temporal alignment of the labeled time series segments.
